(19) United States
(12) Patent Application Publication
(10) Pub. No.: US 2024/0016455 A1
(43) Pub. Date: Jan. 18, 2024

(54) SYSTEMS AND METHODS TO PROTECT AGAINST INFECTIOUS DISEASE

(71) Applicants: ResMed Pty Ltd, Bella Vista NSW (AU); ResMed Inc., San Diego, CA (US)

(72) Inventors: Michael Pinczuk, Sydney (AU); Ian Law, Sydney (AU); Rory Schermerhorn, San Diego, CA (US)

(21) Appl. No.: 18/023,565

(22) PCT Filed: Aug. 30, 2021
(86) PCT No.: PCT/US2021/048230

§ 371 Date: Feb. 27, 2023

Related U.S. Application Data
(60) Provisional application No. 63/072,529, filed on Aug. 31, 2020.

(51) Int. Cl. A61B 5/00; G16H 40/67; G16H 50/30

(52) U.S. Cl. CPC A61B 5/7275; G16H 40/67; G16H 50/30

(57) ABSTRACT

An alert system usable to protect an individual from infectious disease is disclosed. The alert system can make use of one or more body-worn sensors to identify different entities within an environment, as well as their positional relationship (e.g., distance) to a reference location (e.g., the user and/or the alert system). The alert system can present alerts, such as through an augmented reality (AR) display, to identify potential risks and provide information to help minimize risk of infection. The AR display can identify risks with helpful and easy-to-interpret graphics or other overlays. Potential risks can include proximity to other individuals in an environment, contact with high-touch surfaces (e.g., fomites), insufficient cleaning (e.g., insufficient handwashing techniques), and others. The alert system can monitor the environment in real-time to assist the user in preventing transmission of pathogens to or from the user.

### CROSS REFERENCE TO RELATED APPLICATIONS

The present application claims the benefit of, and priority to, U.S. Provisional Patent Application No. 63/072,529, filed Aug. 31, 2020 and entitled “SYSTEMS AND METHODS TO PROTECT AGAINST INFECTIOUS DISEASE”, which is hereby incorporated by reference in its entirety.

### TECHNICAL FIELD

The present disclosure relates to prevention of infectious disease generally and more specifically to augmented reality systems for preventing infectious disease.

### BACKGROUND

Infectious diseases can range from mildly harmful to fatal. While treatments for various diseases are being continuously developed and refined, it is beneficial to provide protection against infectious diseases so the individual does not become ill in the first place. Infectious diseases can be caused by many different types of organisms, such as bacteria, viruses, parasites, and the like.

Different diseases can be transmitted through different routes of transmission, such as aerosol, direct contact, fomite, oral, and vector. Aerosol transmission involves transmitting pathogens contained in aerosol droplets (e.g., droplets entrained in air). Direct contact transmission involves transmitting pathogens, often to open wounds or mucous membranes of a recipient, through direct contact with an infected animal, human, or surface. Oral transmission involves consumption of pathogens, such as those contained in food and water, or licking or chewing contaminated objects (e.g., utensils and cups). Fomite transmission involves transmitting pathogens from a source of the infection to an animal or human via another object (e.g., clothing, door knobs, and the like), often an object that can be passed around or carried from one location to another. Fomite transmission includes other routes of transmission, such as direct contact or oral transmission. Vector transmission involves an organism (e.g., an insect) acquiring a pathogen and then transmitting it to a recipient.

Since pathogens tend to be invisible to the naked eye, it can be difficult for individuals to accurately know, understand, and keep track of the risks of infection during everyday life. As a result, individuals may be needlessly exposed to pathogens or may be overprotective when the risk of infection is sufficiently low. Additionally, individuals may naturally engage in activities with a high risk of infection, such as touching a door knob on an often-used public door, without realizing the risk or without realizing the risk until after the activity.

There is a need for improved techniques for protecting against infectious disease.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a method for protecting against transmission of infectious diseases comprising: collecting sensor data associated with an environment using one or more sensors on a mobile platform associated with a user located within the environment, wherein a location of the user within the environment defines a reference location; identifying a set of entities in the environment using the sensor data, the set of entities including one or more entities; determining a high-risk action associated with the set of entities using the sensor data, wherein the high-risk action is associated with an interaction between the user and at least one entity of the set of entities; measuring a distance, using the sensor data, between the at least one entity of the set of entities and the reference location; generating an alert when the measured distance drops below a threshold distance, wherein the alert is indicative of the high-risk action; and presenting the alert on an interface associated with the mobile platform.

In some cases, presenting the alert includes generating an audio alert or a haptic alert indicative of a direction of the at least one entity from the reference location. In some cases, the at least one entity is a moving entity. In some cases, the at least one entity is a person in the environment.

In some cases, determining the high-risk action includes: determining an infection risk score associated with the person using the sensor data; and identifying that interaction with the person is the high-risk action when the infection risk score is above an infection risk threshold. In some cases, determining the infection risk score includes measuring a temperature of the person and calculating the infection risk score based on the measured temperature. In some cases, determining the infection risk score includes: detecting one or more symptom-related actions based on the sensor data; and calculating the infection risk score based on the detected one or more symptom-related actions. In some cases, the method further comprises updating the threshold distance based on the infection risk score. In some cases, presenting the alert further comprising presenting an indication that the person may be ill. In some cases, determining the high-risk action includes: receiving a communication associated with the person, wherein the communication is indicative of an infection risk associated with the person; and identifying that interaction with the person is the high-risk action based on the infection risk. In some cases, the at least one entity is a surface, and wherein determining the high-risk action includes determining that the surface is a high-touch surface.

In some cases, the method further comprises determining an environmental condition using the sensor data, wherein determining the high-risk action is further based on the environmental condition. In some cases, the method further comprises: determining an environment type of the environment using the sensor data; and setting the threshold distance based on the environment type. In some cases, the method further comprises: determining a geolocation associated with the environment; accessing a rule based on the geolocation; and setting the threshold distance based on the rule. In some cases, the method further comprises: identifying rule signage using the sensor data; determining a rule based on the rule signage; and setting the threshold distance based on the rule. In some cases, the rule signage is a sign present in the environment indicative of a desired amount of distancing between individuals.

In some cases, presenting the alert includes presenting information about reducing a risk of infection after engaging in the high-risk action. In some cases, the information about reducing the risk of infection includes i) an instruction to wash hands; ii) an instruction to sanitize hands; iii) an instruction to replace a facial covering; iv) an instruction to increase the distance between the at least one entity and the reference location; or v) any combination of (i)-(iv). In some cases, the information about reducing the risk of infection includes an instruction to deploy a facial covering. In some cases, the method further comprises: detecting deployment of the facial covering using the sensor data; and ceasing to present the instruction to deploy the facial covering in response to detecting deployment of the facial covering. In some cases, the method further comprises automatically deploying a facial covering when the determined distance between the at least one entity and the reference location drops below the threshold distance. In some cases, the facial covering is mechanically coupled to the mobile platform. In some cases, deploying the facial covering includes moving a face shield from a stowed position to a deployed position, wherein the face shield covers at least a portion of a face of the user when in the deployed position. In some cases, deploying the facial covering includes moving a facemask from a stowed position to a deployed position, wherein the facemask covers a mouth and nose of the user when in the deployed position. In some cases, deploying the facial covering includes generating an air curtain around a portion of a face of the user.

In some cases, the method further comprises: storing information associated with the high-risk action in response to the measured distance dropping below the threshold distance; and generating a summary of detected high-risk actions that occurred within a period of time, wherein generating the summary includes accessing the stored information associated with the high-risk action. In some cases, the period of time is a preset period of time. In some cases, the period of time is defined by a first time and a second time, wherein the first time is associated with the reference location being located in a first geolocation, and wherein the second time is associated with the reference location being located in a second geolocation. In some cases, the summary of detected high-risk actions includes a score based on a count of the detected high-risk actions. In some cases, the summary of detected high-risk actions includes i) a listing of types of high-risk actions associated with each of the detected high-risk actions; ii) a listing of types of entities associated with each of the detected high-risk actions; or iii) a combination of (i) and (ii). In some cases, the types of high-risk actions includes i) touching; ii) approaching; iii) remaining in proximity; or iv) any combination of (i)-(iii). In some cases, the types of entities includes i) a person; ii) a non-person animal; iii) a surface; iv) a region of an environment; v) a face of the user; vi) a facial covering of the user; or vii) any combination of (i)-(vi). In some cases, the detected high-risk actions includes high-risk actions associated with entities not within a predefined cohort of entities. In some cases, the user is a student, wherein the student is part of a pod of individuals, and wherein the predefined cohort of entities is the pod of individuals. In some cases, the method further comprises storing a video clip associated with the high-risk action in response to the distance measurement dropping below the threshold distance.

In some cases, the method further comprises receiving health information using a wireless connection, wherein the health information is associated with the at least one entity of the set of entities, wherein determining the high-risk action associated with the interaction between the user and the at least one entity of the set of entities includes identifying the at least one entity of the set of entities as a high-risk entity using the received health information. In some cases, the cleaning information is associated with at least another entity of the set of entities, wherein determining the high-risk action associated with the interaction between the user and the at least one entity of the set of entities includes excluding the at least another entity of the set of entities based on the received cleaning information. In some cases, the method further comprises: receiving a privacy command; and disabling at least one of the one or more sensors in response to receiving the privacy command.

In some cases, the method further comprises: receiving distance input through an input information associated with the mobile platform; setting the threshold distance based on the distance input. In some cases, the method further comprises dynamically updating a display device using the threshold distance in response to receiving the distance input. In some cases, the display device is an augmented reality display device, and wherein updating the display device includes changing a radius of at least a portion of a circle presented on the display device based on the distance input. In some cases, the at least a portion of a circle overlaid on a region of the display device associated with the at least one entity. In some cases, receiving distance input through the input interface includes detecting a user gesture.

In some cases, the method further comprises: generating an additional alert when the determined distance drops below an additional threshold distance, wherein the additional threshold distance is lower than the threshold distance, and wherein the additional alert is indicative of a higher degree of urgency than the alert; and presenting the additional alert on the interface. In some cases, presenting the alert includes presenting an augmented reality alert on a displace device of the mobile platform, wherein presenting the additional alert includes updating the augmented reality alert to indicate the higher degree of urgency. In some cases, updating the augmented reality alert includes modifying a color of the augmented reality alert.

In some cases, presenting the alert includes presenting an augmented reality alert on display device of the mobile platform. In some cases, the augmented reality alert is overlaid on a region of the display device associated with the high-risk action. In some cases, presenting the augmented reality alert on the display device includes presenting an overlay on a digital image of the environment. In some cases, the display device is an opaque display device that occupies a portion of a field of view of the user. In some cases, the display device is a non-opaque display device that occupies a portion of a field of view of the user.

In some cases, the method further comprises displaying the distance between the at least one entity and the reference location on the display device. In some cases, presenting the augmented reality alert includes presenting at least a portion of an entity ring centered at the at least one entity, wherein a radius of the entity ring is indicative of the threshold distance or a supplemental threshold distance from a center of the at least one entity. In some cases, presenting the augmented reality alert includes presenting at least a portion of a reference ring centered at the reference location, wherein a radius of the reference ring is indicative of the threshold distance or a supplemental threshold distance from a center of the at least one entity.

In some cases, the high-risk activity is associated with particulate projected from the at least one entity, and wherein presenting the augmented reality alert includes indicating the occurrence of the projection of the particulates. In some cases, the method further comprises determining an expected path of travel associated with the particulate projection, wherein indicating the occurrence of the projection of the particulates includes indicating the path of travel associated with the projected particulates.

In some cases, the method further comprises presenting an additional augmented reality alert associated with the at least one entity before the determined distance drops below the threshold distance. In some cases, the additional augmented reality alert is indicative of the high-risk activity. In some cases, the at least one entity includes a moving entity, the method further comprising calculating a probable path of the at least one entity, wherein the additional augmented reality alert is indicative of the probable path of the at least one entity.

In some cases, the method further comprises determining an infection risk score associated the at least one entity, wherein the additional augmented reality alert is indicative that the infection risk score associated with the at least one entity is above a threshold score. In some cases, the additional augmented reality alert is indicative that the at least one entity includes a high-touch surface. In some cases, the additional augmented reality alert is indicative of a need to perform handwashing. In some cases, the additional augmented reality alert is indicative of a percentage of completion of handwashing. In some cases, the additional augmented reality alert includes an overlay associated with hands of the user, wherein the overlay differentiates washed and unwashed portions of the hands of the user.

In some cases, the method further comprises identifying a commercial establishment associated with the environment and accessing a health rating or a cleanliness rating of the commercial establishment, wherein the additional augmented reality alert is based on the health rating or the cleanliness rating of the commercial establishment. In some cases, the method further comprises determining a number of individuals in the environment using the sensor data, wherein the additional augmented reality alert is based on the determined number of individuals. In some cases, the additional augmented reality alert includes an indication that the determined number of individuals exceeds a threshold number of individuals. In some cases, the method further comprises determining an area or a volume of the environment using the sensor data, wherein the threshold number of individuals is based on the determined area or the determined volume of the environment. In some cases, the method further comprises identifying a commercial establishment associated with the environment and accessing an occupancy rule associated with the identified commercial establishment, wherein the threshold number of individuals is based on the accessed occupancy rule.

In some cases, the method further comprises determining a path between the entities of the set of entities using the sensor data, wherein the path is calculated to maintain at least the threshold distance between the path and each entity of the set of entities, wherein the additional augmented reality alert is indicative of the determined path. In some cases, the method further comprises determining a path between the entities of the set of entities using the sensor data, wherein the path is calculated to maximize distance between the path and each entity of the set of entities, wherein the additional augmented reality alert is indicative of the determined path. In some cases, a subset of the set of entities form a queue, and wherein the additional augmented reality alert includes an overlay indicating a point on a floor of the environment that is within the queue or at an end of the queue, wherein the point on the floor is at least the threshold distance spaced apart from a nearest entity within the subset of the entities forming the queue.

In some cases, the at least one entity includes a surface, and wherein determining the high-risk action includes: identifying a type of surface based on the sensor data; determining a pathogen risk score associated with the type of surface; and identifying that interaction with the surface is the high-risk action when the pathogen risk score is above a pathogen risk threshold.

In some cases, the mobile platform is configured to be worn on a head of the user. In some cases, the mobile platform includes a headband supporting a set of haptic feedback devices. In some cases, the one or more sensors include i) a camera; ii) a thermal imager; iii) a rangefinder, or iv) any combination of (i)-(iii).

Embodiments of the present disclosure include a system comprising: a control system including one or more processors; and a memory having stored thereon machine readable instructions; wherein the control system is coupled to the memory, and the method described above is implemented when the machine executable instructions in the memory are executed by at least one of the one or more processors of the control system.

Embodiments of the present disclosure include a system for protecting against transmission of infectious diseases, the system including a control system configured to implement the method described above.

Embodiments of the present disclosure include a computer program product comprising instructions which, when executed by a computer, cause the computer to carry out the method disclosed above. In some cases, the computer program product is a non-transitory computer readable medium.

### BRIEF DESCRIPTION OF THE DRAWINGS

The specification makes reference to the following appended figures, in which use of like reference numerals in different figures is intended to illustrate like or analogous components.

FIG. 1 is a schematic diagram of an alert system for protecting against infectious disease, according to certain aspects of the present disclosure.

FIG. 2 is a schematic diagram illustrating a user wearing an alert system for protecting against infectious disease, according to certain aspects of the present disclosure.

FIG. 3 is an illustration of a field of view of a user including augmented reality overlays illustrating distance-based alerts, according to certain aspects of the present disclosure.

FIG. 4 is an illustration of a field of view of a user including augmented reality overlays illustrating high-touch alerts, according to certain aspects of the present disclosure.

FIG. 5 is an illustration of a field of view of a user including augmented reality overlays illustrating high-risk alerts, according to certain aspects of the present disclosure.

FIG. 6 is an illustration of a field of view of a user including augmented reality overlays illustrating high-risk alerts in a restroom, according to certain aspects of the present disclosure.

FIG. 7 is an illustration of a field of view of a user including augmented reality overlays illustrating alerts associated with cleaning actions, according to certain aspects of the present disclosure.

FIG. 8 is a flowchart depicting a process for protecting against infectious disease, according to certain aspects of the present disclosure.

FIG. 9 is a flowchart depicting a process for determining a high-risk action associated with a person, according to certain aspects of the present disclosure.

### DETAILED DESCRIPTION

Certain aspects and features of the present disclosure relate to an alert system usable to protect an individual from infectious disease. The alert system can make use of one or more body-worn sensors to identify different entities within an environment, as well as their positional relationship (e.g., distance) to a reference location (e.g., the user and/or the alert system). The alert system can present alerts, such as through an augmented reality (AR) display, to identify potential risks and provide information to help minimize risk of infection. The AR display can identify risks with helpful and easy-to-interpret graphics or other overlays. Potential risks can include proximity to other individuals in an environment, contact with high-touch surfaces (e.g., fomites), insufficient cleaning (e.g., insufficient handwashing techniques), and others. The alert system can monitor the environment in real-time to assist the user in preventing transmission of pathogens to or from the user.

As used herein, the term environment can include any suitable space or location, such as an outdoor space (e.g., uncovered field, sidewalk of a street with buildings, a covered gazebo, and others) or an indoor space (e.g., a room, an entryway, a conference room, an airport, a vehicle, and others). In some cases, the environment can be automatically bounded by a preset distance (e.g., anything within 30 feet), a dynamic distance (e.g., a distance set by identification of a type environment), or sensed boundaries (e.g., detected walls, floors, ceilings, and the like).

Certain aspects and features of the present disclosure make use of measuring distances from a user to an entity in an environment or some other object in the environment (e.g., a wall). Distance measuring can be based on a reference location, which can be defined as the location of the user. In some cases, the reference location can be the location of the alert system and/or a location of the one or more sensors of the alert system. Additionally, distances and threshold distances as disclosed herein can be measured and/or calculated form a center or a surface of any two objects. For example, when a six foot distance is determined between the user and an individual (e.g., a person), the six foot distance can be defined by a distance between central axes passing vertically through the user and the individual, by a distance between a surface of the user (e.g., skin) and a surface of the individual, or a distance between a surface of one of the user and individual and a central axes passing vertically through the other of the user and individual (e.g., from a center of the user to a surface of an individual).

The alert system can be incorporated into one or more housings and can be couplable to a user in any suitable fashion. In some cases, the alert system can take the form of a head-worn device, such as a pair of glasses, an attachment to a pair of glasses, and/or a headband. The alert system can acquire information about the environment through one or more sensors, and optionally a network interface (e.g., a wireless connection to another device directly, to a device on an intranet, and/or to a device on the Internet). Examples of suitable sensors are described herein and can include sensors such as cameras (e.g., human-vision cameras, infrared cameras, and the like), microphones, depth sensors, pyrometers, and the like.

The alert system can provide alerts through one or more outputs. Examples of categories of alerts include informational alerts (e.g., providing information about a potential risk) or attention-seeking alerts (e.g., providing an urgent alert about an imminent or currently occurring risk). In some cases, an alert can facilitate reducing the user's risk of infection, such as by providing instructions for taking actions to reduce the user's risk of infection or warning the user of potential or actual increases in the user's risk of infection. In some cases, the alert system can provide an alert as an audible alert (e.g., a tone or message played on a speaker at or near a user's ear). In some cases, the alert system can provide an alert as a haptic alert (e.g., a vibration or tapping sensation generated by a feedback device such as a vibration motor or electromechanical force device). In some cases, the alert system can provide an alert as a visual alert, such as a graphic. In some cases, a visual alert can be provided on a screen, such as a screen of a smartphone. In some cases, the visual alert can be provided as an AR overlay.

An AR overlay can include a graphic (e.g., an icon, a color, an outline, a shape, text, or the like) superimposed on the user's field of view. The AR graphic can be presented to cover an object in the user's field of view by being presented at a location in the user's field of view that would otherwise include the object. AR graphics can be presented in various fashions, including projection-based, native-sight-based, or video-based. A projection-based AR overlay can be a graphic that is projected onto an object in the environment. The projection-based AR overlay can be seen by the user and others in the environment. A native-sight-based AR overlay can be a graphic that is displayed to the user over the user's natural view of the environment, such as via a transparent or translucent display device. A native-sight-based AR overlay may be visible only to the user. A video-based AR overlay can be a graphic that is superimposed on a camera feed (e.g., a live video feed) of the environment, in which case the user's view of the environment occurs via the camera feed. A video-based AR overlay may be visible only to the user.

In some cases, the alert system can provide an alert in the form of AR overlays depicting boundaries around individuals and/or other entities in an environment, such as to indicate a threshold distance from the individual and/or other entity. Such AR overlays can be known as entity rings (for entities in the environment other than the user) or reference ring (for the user), although other shapes can be used. The entity ring or reference ring can include a circle that appears to surround the entity. The circle can be in a plane parallel the ground, but that need not always be the case. The circle can be at ground level (e.g., appearing to be painted on the ground), in a plane parallel the ground that is coincident with the entity (e.g., a plane through a torso of an individual), or at other levels. In some cases, the circle can appear to be painted over multiple surfaces, such as the ground, objects on the ground, and walls. In some cases, entity rings and reference rings can be sized such that the radius of the circle is equal to a desired threshold distance, in which case an entity entering the circle is within the threshold distance. In other cases, however, entity rings and reference rings can be sized such that the diameter of the circle is equal to a desired threshold distance, in which case an entity is within the threshold distance when the entity ring and reference ring overlaps.

In an example, if there is a desire to maintain at least a six-foot distance between individuals for the purpose of reducing risk of pathogenic transmission, the threshold distance can be set at six feet and the AR overlay can take the appearance of a circle around each individual having a radius of six feet. Such an overlay can be quickly and easily interpreted by the user to help the user maintain the recommended distancing. In some cases, such an AR overlay can also be presented around the user, providing a quick and easily interpretable indication of the recommended distancing around the user. When another individual approaches the circle, the user can know whether or not to move or whether or not to ask the individual to stop approaching. In some cases, crossing boundaries of such circles can generate elevated alerts, such as additional AR overlays or a change in the AR overlay of the circle. These elevated alerts can provide a quick and easily interpretable indication that another individual is too close to the user, giving the user an opportunity to increase the distance between the user and the individual. In some cases, other AR overlays can be used to indicate distances, such as lines, arrows, distance measurements (e.g., text indicating distance to an entity), and the like.

In some cases, the alert system can provide an alert in the form of an AR overlay covering (e.g., with a translucent shape), surrounding (e.g., with an outline), or otherwise calling attention to a high-touch surface in the environment. This type of AR overlay can be a surface contact alert. The surface contact alert can alert the user to avoid touching certain surfaces that may pose an increased risk for pathogenic transmission, such as surfaces likely to be touched by others. For example, when entering an establishment through a standard door, a doorknob may be a high-touch surface, since it is likely many people touch the doorknob, whereas all or parts of the door's facade may not be high-touch surfaces because they are not as likely to be touched by others. In some cases, the surface contact alert can be presented in a fashion to make the high-touch surface appear to be highlighted, of a different color, flashing, or otherwise attention-seeking. In some cases, other indications can be made to call attention to the high-touch surface.

In some cases, the alert system can use sensor data to determine a material of a surface (e.g., a high-touch surface or a potential high-touch surface). For example, machine vision or other sensors can be used to identify that a particular surface is made of cardboard, stone, plastic, steel, or other materials. In some cases, the alert system can determine whether a surface is a porous or nonporous surface. In some cases, the alert system can present an alert indicating the material of the surface. In some cases, the alert system can present an alert and/or adjust a parameter of the alert system based on the material of the surface. For example, since the transfer efficiency of pathogens via nonporous surfaces is greater than that for porous surfaces, the alert system may provide an alert or make a determination that a particular surface is a high-touch surface if it is determined to be nonporous, but is not a high-touch surface if it is determined to be porous. In some cases, different materials can have different pathogen risk scores. The alert system can determine that a surface is a high-touch surface if the pathogen risk score associated with the surface is above a pathogen risk threshold. In some cases, the pathogen risk threshold can be preset, such as factory-set (e.g., set during manufacturing), business-set (e.g., set by an employer), group-set (e.g., set by a group, such as a school), or user-selectable. In some cases, the pathogen risk threshold can be dynamically adjusted based on sensor data, such as dynamically adjusted based on the user's location, the type of environment in which the user is located, the number of individuals in the environment, or other such factors.

In some cases, certain alerts (e.g., information alerts) can be presented at all times while the entity associated with the alert is in the environment and/or in the user's field of view. In some cases, different alerts can be presented based on different threshold distances. For example, in some cases, information alerts may only be presented when the entity is within a certain informational threshold distance (e.g., circles indicating safe distances around individuals are only shown for individuals within 20 feet of the user). In another example, an attention-seeking alert may only be presented when an entity is within a warning threshold distance (e.g., a warning appearing when an individual is within 6 feet of the user). In some cases, additional threshold distances can be used for other purposes, such as to provide a series of escalating alerts.

In some cases, threshold distances can be preset (e.g., set by a user) or can be dynamic (e.g., set based to sensor data, such as estimated room size, identified environment type, approximate geolocation, and the like). In some cases, a user can set threshold distances by manipulating a control while the alert system dynamically adjusts the size of the AR overlay (e.g., the circle around the user or around another individual) in real time. The control can be physical (e.g., pressing a physical or digital button on a device) or gesture-based (e.g., making gestures with a hand that are detected by the one or more sensors of the alert system). For example, the user can manipulate a control to increase the threshold distance and see the circle around the user increase accordingly in real time. The alert system can receive distance input as a particular size (e.g., six feet), as an enumerated size (e.g., large), as commands to increase or decrease size, or otherwise.

In some cases, the alert system can monitor behavior of individuals in the environment to trigger alerts and/or adjust parameters of the alert system (e.g., adjust threshold distances). For example, the alert system can analyze sensor data to detect symptom-related actions associated with an individual in the environment. Symptom-related actions can include any action that may be indicative that the individual may be carrying transmissible pathogens. Examples of such symptom-related actions can include sneezing, sniffles, runny noses, coughing, exhibiting a fever (e.g., as measured by a pyrometer), and the like. In some cases, threshold distances can be automatically adjusted based on the detection of symptom-related actions (e.g., the presence of, intensity of, type of, and/or frequency of symptom-related actions). In some cases, when symptom-related actions are identified, an alert can be provided to a user notifying the user that the individual in question may be ill. For example, a user wearing the alert system around family members may be able to preemptively identify a potential illness in the family member when the alert system detects symptom-related actions and provides a relevant alert. In some cases, the alert system can provide an AR overlay indicating the intensity, extent, directionality, or other information associated with the symptom-related action. For example, when the alert system detects an individual sneezing, the alert system can present an AR overlay indicating the direction and/or extent of the sneeze (e.g., a region indicating the trajectory of the sneeze and/or where particulates from the sneeze may reach). As an example, the AR overlay can take the form of a translucent or transparent plume shape extending from the individual's mouth for a suitable distance (e.g., approximately six feet).

In some cases, the alert system can monitor the user's actions or behaviors to trigger alerts. For example, the alert system can analyze sensor data to determine that the user is moving the user's hand towards the user's face. To discourage face touching, which can go unnoticed by the user and can promote transmission of pathogens into the user's body, the alert system can present an alert. For example, the alert system can present an AR overlay and/or another alert (e.g., an audio alert or haptic alert) that indicates the user is engaging in a potentially unsafe activity or that recommends the user not touch the user's face. In some cases, the alert system can provide an alert after the user's action (e.g., after the user touched their face with their hands) in the form of a notice informing the user that the action occurred and optionally recommending against it in the future. In some cases, the alert system can track the number of times such actions occurred to present summary information to the user at the end of a day or for other purposes.

In another example, the alert system can monitor the user performing high-risk interactions with the environment, such as touching a high-touch surface (e.g., a public doorknob), coming in close proximity to another individual (e.g., standing close to a stranger in a public space), or remaining in enclosed public spaces for long durations (e.g., staying in a store for longer than a threshold duration). High-risk interactions can be any interactions having a likelihood (e.g., a strong likelihood) of transferring pathogens to the user. Categorization of an interaction as a high-risk interaction can be based on user preference and/or model training, as disclosed herein. The alert system can present alerts before, during, or after the identified high-risk interaction. For example, as the user reaches out to touch a high-touch surface, the alert system can provide an alert to notify the user of the unsafe action and/or discourage the action.

In some cases, after engaging in a high-risk interaction, the alert system can provide ongoing alerts to remind the user that the user may need cleaning to reduce the risk of pathogenic transmission. For example, after the alert system detects the user has touched a high-touch surface, the alert system can provide an alert in the form of a warning notifying the user to wash their hands. This alert can remain until the alert system detects that the user has washed their hands or until the user dismisses the warning. In some cases, the warning can be an AR overlay in the form of text, a graphic, or highlighting over the user's hands representing the regions of the user's hands that may have been exposed to pathogens from the high-risk interaction. In another example, after coming too close to an individual, the alert system can provide an alert in the form of a warning that recommends the use take a shower or perform other cleaning activities (e.g., changes clothes).

In some cases, the alert system can be used to detect and/or facilitate handwashing. The alert system can use sensor data to identify the user's hands during a handwashing procedure and monitor the duration and/or extent of handwashing. In some cases, the alert system can provide an alert in the form of a timer, reminding the user to continue the handwashing procedure for a preset duration (e.g., twenty seconds). In some cases, the alert system can provide an AR overlay that highlights regions of the user's hands that have or have not been washed. Thus, the user can continue washing hands until their hands are fully covered or not at all covered by the AR overlay, respectively. This AR overlay can be in the form of a translucent or opaque shape bounded by the outline of the hands and the regions determined to be washed or not washed. Other forms for the AR overlay can be used. When washing hands using the alert system, the user can quickly identify any regions of the hands that the user may have inadvertently missed or inadvertently did not clean sufficiently (e.g., a region of the hand may only be identified as being washed if it is cleaned for a threshold duration of time, such as five seconds). As another example, a user washing their hands using a hand sanitizer can be monitored by the alert system, which can provide alerts indicating regions of the hands that were not sufficiently washed by the hand sanitizer (e.g., the hand sanitizer did not reach the region or the hand sanitizer was not left for a sufficient length of time).

In some cases, the alert system can likewise be used to detect and/or facilitate cleaning objects other than the user's hands. The alert system can provide similar alerts (e.g., AR overlays of timers and/or shapes indicating cleaned or not cleaned regions) on objects in the user's environment. For example, for a user cleaning a table, the alert system can provide a timer indicating a remaining time the user should leave a cleaning solution before wiping the cleaning solution away, and/or the alert system can provide an AR overlay in the form of a shape covering regions of the table that have or have not been sufficiently cleaned.

In some cases, the alert system can be used to provide alerts indicating the level of cleanliness of an environment or object in an environment. The level of cleanliness can be based on environmental measurements (e.g., particulates in the air from an air sensor data, visible matter from a camera feed, and the like), cleaning action tracking (e.g., determining that a cleaning action is occurring by the user or an individual in the environment and tracking interactions with the cleaned object and/or time since the cleaning action occurred), reception of remote data (e.g., receiving data from a remote device indicating level of cleanliness for a particular object and/or environment), or any combination thereof. Remote data can include cleaning information associated with cleaning actions taken in one or more environments. The cleaning information can indicate that an entity (e.g., a surface) in an environment has been cleaned. As an example, employees of a business may wear alert systems as disclosed herein. In response to someone cleaning a conference room, a signal can be sent to the alert systems of the employees identifying the conference room and indicating the time of cleaning. Thereafter, when employees approach the conference room, their individual alert systems can present alerts associated with the estimated cleanliness of the conference room, such as based on the number of people who have entered the conference room and/or an elapsed time since the cleaning occurred. For example, an employee approaching the conference room within the first 20 minutes of the cleaning may see an AR overlay that covers the handles of the conference room door in a green or blue color, indicating clean, whereas an employee approaching the conference room five hours after the cleaning may see an AR overlay that covers the handles of the conference room door in an orange or red color, indicating unclean. In some cases, such alert systems can be used by cleaning staff to quickly and efficiently identify surfaces that may need to be cleaned, especially when cleaning protocol requires certain environments and/or objects in environments to be cleaned on a regular basis (e.g., hourly).

In some cases, the alert system can be used to aid in queuing. The alert system can detect a queue location, such as through detection of individuals in a queue (e.g., a group of individuals standing in a line or chain) or detection of queue-prompting elements in the environment (e.g., rope lines, lanes, teller windows, and the like). When a queue location is detected, the alert system can determine a location that is a threshold distance away from an end of the queue and then present an AR overlay marking that location. For example, in a queue of individuals, the alert system can find the end of the queue and present a target graphic as an AR overlay at a location on the floor six feet away from the last member of the queue.

In some cases, the alert system can be used to detect high-traffic areas in an environment. High-traffic areas can be detected based on entity movement within the environment (e.g., monitoring multiple individuals passing through the same area) or can be inferred based on the environment (e.g., the sole doorway in an enclosed space may be inferred to be a high-traffic area since all individuals must pass through that doorway). When a high-traffic area is detected, the alert system provides an alert, such as an AR overlay indicating the detected area is a high-traffic area. Other alerts can be provided.

In some cases, the alert system can be used to detect low-risk areas of an environment. Low-risk areas can be detected based on entity movement (e.g., a region of the environment that individuals tend to avoid) within the environment or can be inferred based on the environment (e.g., the presence of open windows, open doors, or air registers can be indicative of airflow that may lower risk of infection). The alert system can provide an AR overlay in the form of a message recommending that the user move to a low-risk area and/or an AR overlay in the form of a translucent or transparent shape covering or otherwise calling attention to the low-risk area. In some cases, a high-traffic area can be color coded in red, orange, or yellow, and a low-risk area can be color coded in blue or green.

In some cases, the alert system can give environment-specific alerts and/or adjust parameters of the alert system (e.g., threshold distances) based on one or more environmental conditions, such as information about the environment (e.g., a type of environment or measurements of the environment). The alert system can use sensor data to identify the type of environment (e.g., through camera data, audio data, geolocation data, and the like) and present an alert associated with that environment type. Identifying the type of environment can include making an inference based on local data (e.g., using camera data to identify an environment as most likely being an elevator) or with the use of remote data (e.g., using geolocation and/or camera data in combination with a remotely accessible database to identify the environment as a particular environment having an environment type). For example, if the alert system identifies an environment as being an elevator, the alert system can provide an alert in the form of an AR overlay of text recommending that the user only enter the elevator alone or with a maximum number of other individuals. As another example, if the alert system identifies an environment as being an enclosed space, the alert system can provide an alert in the form of an AR overlay of text recommending that the user not linger in the enclosed space.

In some cases, the alert system can obtain information about the environment from the one or more sensors, such as measurements associated with the environment. Measurements of the environment can include measurements based on any suitable sensor data. For example, the alert system can measure distances within a space, such as distances to walls and/or other surfaces, such as to determine approximate square footage and/or approximate volumes of spaces. In some cases, the alert system can measure or otherwise obtain atmospheric data about the environment, such as temperature, humidity, wind speed, air pressure, particulate concentration (e.g., air cleanliness), and the like. For example, if the particulate concentration in the air in an environment is above a threshold, the alert system may present an alert indicating this condition to the user and/or may adjust the threshold distances or threshold lingering time while the user remains in that environment and the particulate concentration remains above the threshold.

In some cases, the alert system can receive a communication from a remote device via a wireless connection (e.g., via a direct wireless connection or via wireless connection through a network) associated with one or more individuals in the environment. For example, a user in an environment may see an individual who is also wearing a similar alert system. The individual's alert system may transmit information to the user's alert system in the form of an information signal. The communication can contain identification information and payload information. The identification information can be used by the user's alert system to identify the individual's alert system. For example, identification information can include nearby wireless signal strengths, geolocation information, or the like. The payload information can include information usable by the user's alert system to generate an alert and/or modify a parameter of the user's alert system. For example, if the individual personally prefers an eight-foot threshold distance instead of a six food threshold distance, the user's alert system, upon receiving the information signal from the individual's alert system, can adjust the threshold distance for that individual when that individual is in the user's field of view (e.g., the circle indicating the threshold distance around that individual would now have an eight foot radius). As another example, the payload information can include health information, such as an indication that the individual is or may be ill, in which case the user's alert system can use that indication to present an alert to the user indicating that the individual may be ill.

In some cases, the alert system can obtain information from remote servers and use that information to present alerts and/or adjust parameters of the alert system. In some cases, the alert system can access a database containing ratings (e.g., cleanliness ratings, health ratings, or other ratings) for a particular business or establishment (e.g., a restaurant). The alert system can then present alerts based on these ratings, such as presenting an AR overlay in the form of a cleanliness meter ranging from zero to 100, with a needle pointing to the cleanliness of the establishment based on the ratings. For example, when walking down a street with multiple restaurants, the user may see two restaurants in their field of view, and the alert system can present AR overlays in the form of cleanliness meters near the doors or signs of each restaurant showing their respective cleanliness levels (or other ratings). Thus, the user is able to quickly and easily see information that may be important to the user in selecting which restaurant to patronize.

In some cases, the alert system can access a database containing rules, such as user-defined rules, cohort-defined rules (e.g., rules for members of the cohort, such as family-specific rules for all family members or company-specific rules for all employees of the company), business-defined rules (e.g., a place of business's own rules, such as a restaurant that wants to keep space between occupants of at least eight feet), laws (e.g., state or federal laws), and/or regulations (e.g., local, state, or federal regulations or other regulations). Rules can establish certain parameters for different environments, which can be leveraged by the alert system to provide alerts and/or update parameters of the alert system (e.g., threshold distances). The alert system can use sensor data (e.g., geolocation, determination of environment type, or others) and the rule database to present an alert to the user and/or adjust a parameter of the alert system.

In some cases, the alert system can use sensor data to identify one or more individuals in the environment. Identifying the one or more individuals can include uniquely identifying the individual and/or identifying the individual as belonging to a particular cohort (e.g., a group of people between which contact is permitted or otherwise having lower risk to transmit new diseases, such as a family living together or a group of students). Based on the identification of the individual, the alert system can provide an alert and/or adjust parameters of the alert system. For example, if the alert system identifies an individual as belonging to a cohort shared by the user (e.g., the same family living together, a group of classmates sharing class spaces, a group of employees sharing working space, or the like), the alert system can ignore or not present alerts that may otherwise be presented, such as not presenting a warning alert when the individual approaches the user and comes within the threshold distance. Cohorts can be predefined (e.g., user-selected) or dynamically created (e.g., the alert system can identify individuals that the user spends time with and automatically add the individual to the user's cohort or present an option to do so). In another example, a student user wearing an alert system can be warned (e.g., using an AR overlay) when approaching another student who is not part of the student user's cohort.

In some cases, the alert system can track events, such as the number of high-risk actions engaged in by the user, the number of high-risk individuals the user interacts with, and/or the number of high-touch surfaces the user interacts with. Such tracking can occur over a predefined period of time (e.g., daily, from sunrise to sunset and sunset to sunrise, from midnight to noon and noon to midnight) or dynamic periods of time (e.g., a period of time when the user is located at a particular geolocation, such as work; a period of time between when the user leaves a first geolocation, such as home, and arrives again at the first geolocation; a period of time the user spends in a particular type of environment). For tracking purposes, periods of time can be continuous (e.g., continuous between a defined start and stop time) or non-continuous (e.g., having multiple sub-periods with their own start and stop times within an overall period start time and stop time).

In some cases, the alert system can generate and/or present a display indicating tracked events, such as a listing of tracked events, a time-lapse visualization of tracked events, and/or a summary of tracked events. For example, at the end of a day, the alert system can generate a message (e.g., in an email, displayed as an AR overlay, and/or transmitted to a remote device such as a smartphone) that indicates the user has touched seven high-touch surfaces, interacted with three individuals within the warning threshold distance, and came near eighty-four individuals outside of the threshold distance. The alert system can also provide feedback and/or guidance to the user based on the tracked events, such as recommendations to avoid touching high-touch surfaces, to wear gloves, to wash hands, or to take a shower.

In some cases, the summary can include i) a listing of types of high-risk actions associated with each of the detected high-risk actions; ii) a listing of types of entities associated with each of the detected high-risk actions; or iii) a combination of (i) and (ii). In some cases, the types of high-risk actions includes i) touching; ii) approaching; iii) remaining in proximity; or iv) any combination of (i)-(iii). In some cases, the types of entities includes i) a person; ii) a non-person animal; iii) a surface; iv) a region of an environment; v) a face of the user; vi) a facial covering of the user; or vii) any combination of (i)-(vi).

In some cases, the alert system can use tracked events to present a score, such as a cleanliness score or a compliance score (e.g., compliance with practices to minimize or reduce risk of pathogenic transmission). The score can be used to evaluate how well the user performed in minimizing risk of pathogenic transmission. In some cases, the score can be a count of the number of detected high-risk actions (e.g., actions that occurred). In some cases, the score can be compared with others to compete for higher ranking on a leaderboard.

In some cases, tracking an event can include storing information associated with the event. Such information can include the type of high-risk action involved, date information, time information, location information, environmental information, and sensor data. In some cases, sensor data can include a photo and/or video clip associated with the event (e.g., leading up to, including, and/or after the event). In some cases, such information can include information usable to identify one or more individuals involved in the event, including or other than the user. The stored information associated with the event can be accessed at a later time to facilitate contact tracing. For example, if the user becomes ill, the stored information can be accessed to help pinpoint one or more events where pathogenic transmission was likely or most likely. This information can help identify the source of the pathogenic transmission to the user, as well as any destinations for pathogen transfer where the user is the source (e.g., individuals with whom the user shook hands after shaking hands with an individual carrying the pathogen).

In some cases, the alert system can be used to generate recommended paths through an environment. The alert system can identify high-risk entities (e.g., high-risk individuals and high-touch surfaces) in an environment and then generate a path from the user to a destination location. Generating the path to the destination can include maintaining maximum distance between the path and the high-risk entities, or at least maintaining maximum distance up to a threshold distance between the user and the high-risk entities. The path, once generated, can be presented to the user, such as via an AR overlay. In some cases, generating the path can include predicting paths of moving entities (e.g., individuals, animals, vehicles, and the like) in the environment and generating the path to avoid the moving entities and/or the paths of the moving entities. By tracking relative movement of the user with respect to the moving entities, the alert system can dynamically update the path to ensure the user avoids the moving entities and/or the paths of the moving entities. For example, a user's path may cross the path of another individual, but at a time when the alert system predicts the other individual would have already passed that location, but if the user starts walking faster such that the user is on track to move too close to the other individual, the alert system may dynamically update the path to ensure sufficient distance is maintained.

The user can provide the destination location through any suitable technique. In some cases, the user can provide the destination location by inputting an address, coordinates, or other location identifiers into a user interface (e.g., via a smartphone communicatively coupled to the alert system). In some cases, the user can manipulate controls of the alert system and/or make gestures detectable by the alert system to move and set a destination marker presented as an AR overlay. For example, the user can make a recognizable and detectable gesture to start a pathfinding function and cause the alert device to present an AR overlay in the form of a hovering destination pin, move their hand until the destination pin rests over the desired destination, and make another gesture to set the pin in place, causing the alert system to generate and then present a path from the user to the destination.

In some cases, the alert system can be used to facilitate deployment of prophylactic devices. Examples of prophylactic devices can include gloves (e.g., surgical gloves) and face coverings (e.g., facemasks and face shields). Facilitating deployment of prophylactic devices can include providing alerts to remind a user to deploy the prophylactic device (e.g., a warning to don gloves or lower a face shield) or actively deploying the prophylactic device (e.g., automatically actuating a face covering). The alert system can facilitate deployment of prophylactic devices upon detecting a high-risk activity, such as proximity of a high-risk individual within a threshold distance, proximity of a high-touch surface within a threshold distance, the user's hand reaching towards a high-touch surface, or the occurrence of a symptom-related action associated with a nearby individual. For example, when approaching a stairwell, the alert system can provide a message, such as via an AR overlay, reminding the user to wear gloves before touching the handrail. As another example, when entering an enclosed space with multiple people, the alert system can provide a warning to wear a facemask. Such a warning can remain and optionally grow in intensity until the alert system detects the user has donned the facemask or the user self-reports donning of the facemask. In other words, the alert system can use sensor data to detect when a facial covering has been deployed (e.g., donned by a user or automatically deployed), then cease presenting a warning to wear the facial covering. As another example, when standing near a sneezing individual, the alert system can detect the sneeze (e.g., through facial expressions that anticipate the sneeze or through the actual sneeze itself) and automatically deploy a face covering to protect the user. In an example, the face covering can be an air curtain or air blade (e.g., pressurized air expelled from one or more nozzles) near the user's face that directs air and particulates away from the user's face. In some cases, an automatically deployable facial covering can be a facemask that is movable between a stowed position and a deployed position, a face shield that is movable between a stowed position and a deployed position, an air curtain, or any combination thereof.

In some cases, the alert system can include a privacy mode, which disables some or all of the one or more sensors. For example, the privacy mode may be desirable when a user is entering a bathroom or bathroom stall. The privacy mode can be enabled when a privacy command is received, such as via a user interface (e.g., a physical or digital button) or via gesture control (e.g., detection of a user's gesture through the one or more sensors of the alert device). In some cases, the privacy mode can be automatically enabled (or an option to enter privacy mode can be presented) based on sensor data, such as a geolocation determined by sensor data or a type of environment (e.g., a bathroom) determined by sensor data.

Certain aspects and features of the present disclosure can make use of machine learning to help detect, identify, and/or classify information (e.g., entities, entity types, material types for surfaces, environments, environment types, individuals, cohort information, hands and handwashing actions, symptom-related actions, and others) based on sensor data from the one or more sensors of the alert system. Machine learning can include training one or more models (e.g., a deep neural network) to perform the functions disclosed herein. In some cases, especially when the sensor data used includes camera data, the sensor data can be applied to a convolutional neural network (CNN) and/or a recurrent neural network (RNN) to help extract the desired information from the sensor data, especially when analyzing sensor data in real-time. Deep neural networks can be trained on data acquired through use of a device containing some or all of the sensor(s) of the alert device.

In some cases, a user can select between using different neural networks. For example, the alert system can allow a user to select between a first neural network designed and trained to identify high-risk individuals, a second neural network designed and trained to identify high-touch surfaces, a third neural network designed to identify cleaning actions, and a fourth neural network designed to identify any combination of high-risk individuals, high-touch surfaces, and cleaning actions. In some cases, the alert system can automatically switch between different neural networks based on geolocation, time of day, environment type, or other such variables.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

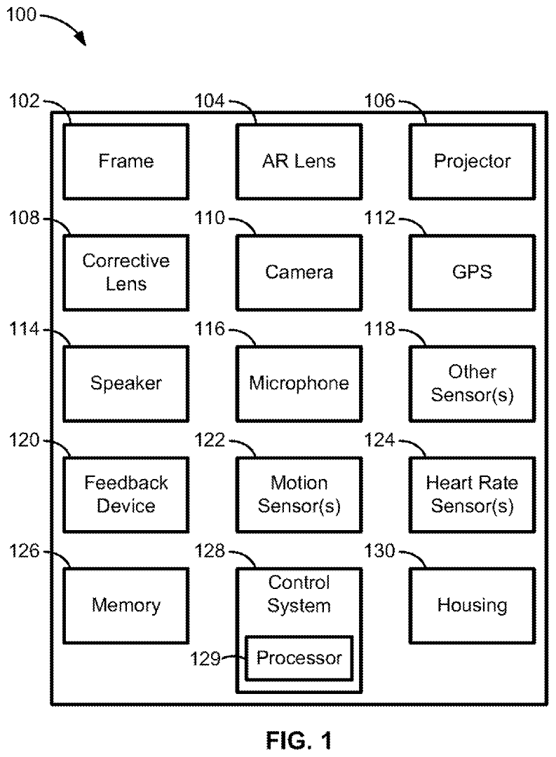

FIG. 1 is a schematic diagram of an alert system 100 for protecting against infectious disease, according to certain aspects of the present disclosure. The system 100 can include a frame 102, an augmented reality (AR) lens 104, a projector 106, a corrective lens 108, a camera 110, a global positioning system (GPS) sensor 112, a speaker 114, a microphone 116, at least one other sensor 118, a feedback device 120, a motion sensor 122, a heart rate sensor 124, a memory 126, a control system 128, a housing 130, or any combination thereof. In some cases, system 100 can include additional components. Components of system 100 can be located in a single housing or spread across multiple housings.

The frame 102 is a structural element designed to secure the system 100 to a user. In some implementations, the frame 102 is an eyepiece frame (e.g., a glasses frame), a watch strap/band, a head gear/strap, etc. or any other element that can be used to secure one or more objects to a user. In some cases, the frame 102 is coupled to a housing 130. The housing 130 mechanically couples to the frame 102 through connecting elements. In some cases, the AR lens 104, the projector 106, the corrective lens 108, the camera 110, the global positioning system (GPS) sensor 112, the speaker 114, the microphone 116, the at least one other sensor 118, the feedback device 120, the motion sensor 122, the heart rate sensor 124, the memory 126, and the control system 128 are located on and/or in or otherwise coupled to the housing 130. In some other implementations, any combination of these elements can be located on, in, and/or otherwise coupled to the frame 102 directly and/or indirectly. In some cases, there may be more than one of any of the following sensors: the at least one other sensor 118, the motion sensor 122, and the heart rate sensor 124. In some cases, the housing 130 is readily removably coupled to the frame 102. In other examples, the housing 130 is not readily removably coupled (e.g., permanently coupled) to the frame 102 such that, for example, removal of the housing 130 requires a breaking of the frame 102 and/or the housing 130.

The AR lens 104 is or includes a prism. In some cases, the AR lens 104 is positioned so as to direct electromagnetic radiation from the projector 106 towards the corrective lens 108. In some cases, the AR lens 104 transmits electromagnetic radiation through the corrective lens 108 away from a user. In other examples, the AR lens 104 transmits electromagnetic radiation off of the corrective lens 108 and towards the user (e.g., towards an eyeball of the user).

The corrective lens 108 is coupled to the frame 102 and configured to be positioned in front of the eye/eyeball of a user. In some cases, the corrective lens 108 provides visual assistance to the user. In other examples, the corrective lens 108 is a plano lens with a power of zero.

The control system 128 is communicatively coupled to the projector 106, the camera 110, the GPS sensor 112, the speaker 114, the microphone 116, the at least one other sensor 118, the feedback device 120, the motion sensor 122, the heart rate sensor 124, and the memory 126. The control system 128 is configured to instruct these various elements to collect data, according to their various characteristics. The control system 128 can further provide for storing the collected data in the memory 126, and/or transmitting the collected data to an external computing device. In some cases, the at least one other sensor 118 is a GPS sensor configured to locate system 100 (and thereby, locate a user associated with system 100). In other examples, the at least one sensor 118 is a depth sensor (e.g., rangefinder) configured to measure a distance of an object, in the field of view of the user, from the housing 130. Suitable depth sensors (e.g., rangefinders) include light detection and ranging (LiDAR) sensors, radio frequency sensors, radar sensors, acoustic sensors, and the like. In some examples, the at least one sensor 118 is a pyrometer for measuring temperature.

The projector 106 is configured to emit electromagnetic radiation in response to instructions from the control system 128. The projector 106 is configured to emit electromagnetic radiation that presents to the user as a graphic, which can be text, an image, a game, or any other visual display. In some cases, the projector 106 sends electromagnetic radiation directly towards the retina of a user. In some cases, the projector 106 is and/or includes a low-intensity laser configured to emit visible light.

While referred to as a “projector,” the projector 106 can take various forms, such as a video screen. For example, in some cases an AR lens 104 can be excluded from system 100 and the projector 106 can take the form of one or two video screens directing light towards the user's eyes. In such a case, the projector 106 can project AR graphics overlaid on a live video feed, such as a live video feed of directly in front of the user collected by camera 110.

The camera 110 is configured to record one or more images and/or video data, including, for example, one or more video clips. In some cases, the camera 110 is positioned on the frame 102 to be substantially aligned with an optical axis of the corrective lens 108. The microphone 116 is configured to record audio data. The control system 128 provides for starting and stopping recording of the camera 110 and/or the microphone 116. The speaker 114 is configured to emit audio data in response to instructions from the control system 128. In some implementations, the speaker 114 and the microphone 116 operate in tandem to provide an auditory interface for a user. Such an auditory interface can receive audio from a user via the microphone 116, process the audio data at the control system 128, determine an auditory response based on the audio data, and provide the auditory response via the speaker 114.

The system 100 further includes a plurality of sensors configured to collect data associated with a user of the system 100. Although particular sensors are shown in FIG. 1, any biometric sensors can be included in the system 100 (for example, the other sensor(s) 118). In some cases, one or more of the sensors can be used to detect movement of a specific body part of a user. For example, the one or more sensors can detect movement of the user's hand, such as movement of the user's hand towards the user's face or towards a high-risk surface.

The system 100 can further include the motion sensor 122, configured to measure motion of system 100. When the system 100 is mounted on the head of a user, the motion sensor 122 generates motion data related to movement of the head of the user. For example, the control system 128 determines a user is turning to look in a different direction, based on data from the motion sensor 122. In some cases, the motion sensor 122 is an accelerometer or a gyroscope.

The system 100 can additionally include the heart rate sensor 124, configured to measure the heart rate of a user and generate heart rate data. In some implementations, the heart rate data indicates (1) a heart rate of the user, (2) a variability of the heart rate of a user between breathing in and breathing out, or (3) both the heart rate and the variability of the heart rate while breathing. In some cases, heart rate data can be used to monitor the user for indications of symptoms.

The system 100 can include a feedback device 120. The feedback device 120 can provide haptic feedback to the user, such as in the form of vibrations or taps. The feedback device 120 can be used to alert the user to the presence of a new alert being displayed by projector 106 or some other alert. In some cases, feedback device 120 can include a number of individually addressable haptic generators located at different points around the user (e.g., vibrating micro motors around the circumference of a headband), which can be individually activated to indicate directionality of an alert. For example, an individual determined to be high-risk approaching from a user's right side can cause the haptic generators on that side of the user to be activated to help the user identify from which direction the high-risk individual is approaching. As the user turns towards the high-risk individual, haptic generators no longer in the direction of the high-risk individual can turn off (e.g., generators on the far right side of the user) and generators in the direction of the high-risk individual can turn on (e.g., generators on the front of the user).

In some implementations, the system 100 is and/or includes a watch, a pair of glasses, a smart phone, and/or is embedded into an article of clothing of a user (e.g., a headband, a hat, a shirt, pants, shorts, etc., or any combination thereof). Therefore, the system 100 is capable of collecting user data and providing instructions to the projector 106 based on the data collected. Additional system examples and methods of providing instructions to the projector are discussed further herein.

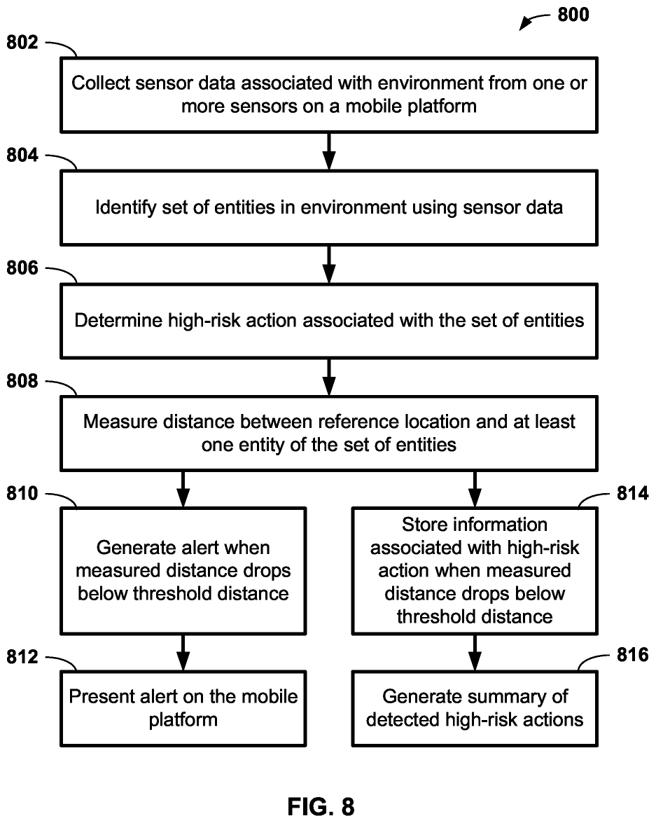

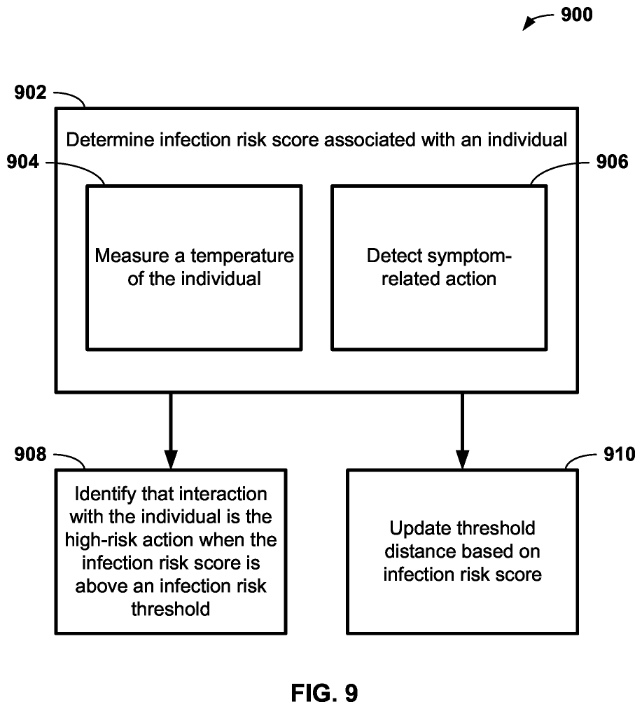

System 100 can include a control system 128. Control system 128 can include one or more processor(s) 129 that can execute instructions to perform certain aspects and features of the present disclosure. Control system 128 can include computer-readable medium, which can be any medium that participates in providing instructions to processor(s) 129 for execution, including without limitation, non-volatile storage media (e.g., optical disks, magnetic disks, flash drives, etc.) or volatile media (e.g., SDRAM, ROM, etc.). The computer-readable medium (e.g., storage devices, mediums, and memories) can include, for example, a cable or wireless signal containing a bit stream and the like. However, when mentioned, non-transitory computer-readable storage media expressly exclude media such as energy, carrier signals, electromagnetic waves, and signals per se. In some cases, computer-readable medium can include various instructions for implementing an operating system and applications, such as computer programs. The operating system can be multi-user, multiprocessing, multitasking, multithreading, real-time and the like. The operating system can performs basic tasks, including but not limited to: recognizing input from an input device (e.g., any of the sensors of system 100); sending output to display device (e.g., projector 106); keeping track of files (e.g., stored sensor data or other data) and directories on computer-readable medium; controlling peripheral devices (e.g., speaker 114 and feedback device 120), which can be controlled directly or through an input/output controller; and managing traffic on a bus coupling electrical components of system 100. The control system 128 can include various instructions for implementing any of processes described herein, including at least processes 800 and 900 of FIGS. 8 and 9, respectively.

The described features can be implemented advantageously in one or more computer programs that are executable on a programmable system including at least one programmable processor (e.g., processor 129) coupled to receive data and instructions from, and to transmit data and instructions to, a data storage system, at least one input device (e.g., camera 110), and at least one output device (e.g., projector 106). A computer program is a set of instructions that can be used, directly or indirectly, in a computer to perform a certain activity or bring about a certain result. A computer program can be written in any form of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a memory, such as a read-only memory, a random access memory, or both. The processor and the memory can be supplemented by, or incorporated in, ASICs (application-specific integrated circuits).

The features can be implemented in a computing system that includes a back-end component, such as a data server, or that includes a middleware component, such as an application server or an Internet server, or that includes a front-end component, such as system 100, or any combination thereof. The components of the system can be connected by any form or medium of digital data communication such as a communication network. Examples of communication networks include, e.g., a local area network, a wide area network, a personal area network, an intranet, and the computers and networks forming the Internet.

The computing system can include clients (e.g., system 100) and servers. A client and server are generally remote from each other and typically interact through a network. The relationship of client and server arises by virtue of computer programs running on the respective computers and having a client-server relationship to each other.

One or more features or steps of the disclosed embodiments can be implemented using an application programming interface (API). An API can define one or more parameters that are passed between a calling application and other software code (e.g., an operating system, library routine, function) that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, communications capability, and the like.

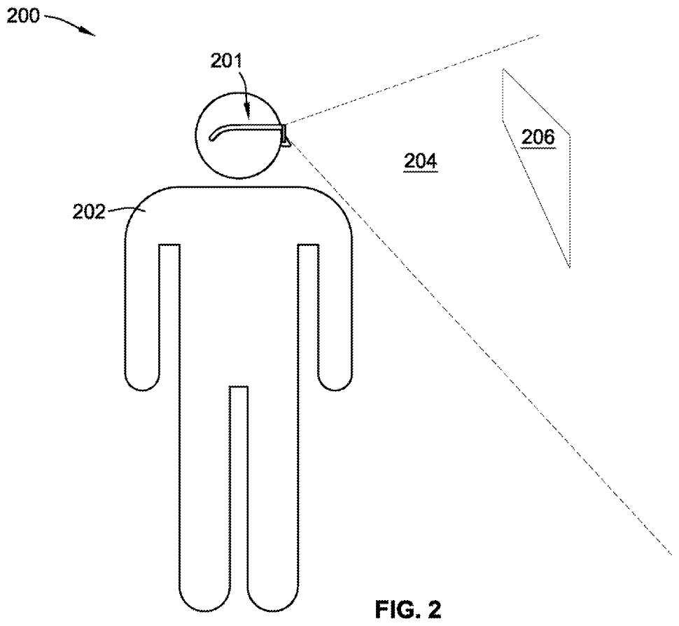

FIG. 2 is a schematic diagram illustrating a user 202 wearing an alert system 200 for protecting against infectious disease, according to certain aspects of the present disclosure. The alert system 200 can be any suitable alert system, such as alert system 100 of FIG. 1. The alert system 200 can be worn on the head of user 202. The alert system 200 can be considered a mobile platform. In some cases, alert system 200 takes the form of a pair of glasses 401, although that need not always be the case. In some cases, the alert system 200 can take the form of a headband, a glasses attachment, a clip, a hat, a head covering, a pair of headphones, a necklace, a collar, an earpiece, or other such wearable devices. In some cases, components of alert system 200 can be spread across a number of housings.

Alert system 200 can track the field of view 404 of the user 202, such as through the use of one or more sensors to track eye movement and/or head movement. In some cases, the alert system 200 can be fixed to the head of a user 202 (e.g., worn as a pair of glasses) such that the alert system 200 can track orientation through one or more motion sensors and/or other sensors.

The alert system 200 can affect the field of view 204 of the user 202 by presenting one or more graphics 406 (e.g., overlays or graphical overlays). Any suitable graphic 406 can be presented, such as an image, text, a region of highlighting, a border, a timer, an alert, instructions, and the like. The graphic 406 can update according to movement of the field of view 204 (e.g., a square highlighting an object in the field of view 204 can grow as the user approaches the object to account for the object appearing larger in the field of view 204) or can remain static in the field of view 204 (e.g., a graphic displaying the current time may always appear in the same position within the field of view 204, such as near the center-top of the field of view 204). In other words, the graphic 406 can appear to the user 202 as fixed with respect to the field of view 204 of the user 202 (e.g., a “static” graphic) or can appear to the user 202 as fixed with respect to the environment or an object in the environment in which the user 202 is located (e.g., a “dynamic” graphic).

In an example, alert system 200 can present a graphic 406 indicating a zone of safety around the user 402. This graphic 406 can appear as a circle (e.g., a circle 6′ in radius) around the user 402 as the user changes their field of view 404. In another example, a graphic 406 can include a text indication of the distance between the user 202 and another individual, such as “10 feet.” In another example, a graphic 406 can include a target that appears on the ground at a location a safe distance away from the last person in a queue, indicating where the user 202 should stand to join the queue. Other examples are discussed herein.

As discussed in further detail herein, a graphic 406 can be used to provide alerts and other information based on the user's proximity to any entity (e.g., persons, surfaces, objects, and the like) in an environment. The alert system can process sensor data to identify the entities in an environment and then identify high-risk activities that can occur between the user and the entities. For example, since being in close proximity to another individual can be a high-risk activity due to the increased likelihood of pathogenic transmission (e.g., via an aerosol route of transmission), the alert system can identify a number of individuals out of the various entities in the environment, then provide alerts when the distance from the user to each individual drops below a threshold amount. In another example, since touching high-touch surfaces (e.g., a public handrail) can be a high-risk activity, due to the increased likelihood of pathogenic transmission (e.g., via a fomite route of transmission), the alert system can identify a number of high-touch surfaces out of the various entities in the environment and provide alerts to indicate that each high-touch surface is a high-touch surface (e.g., with a red or flashing overlay) when the user approaches the high-touch surface.

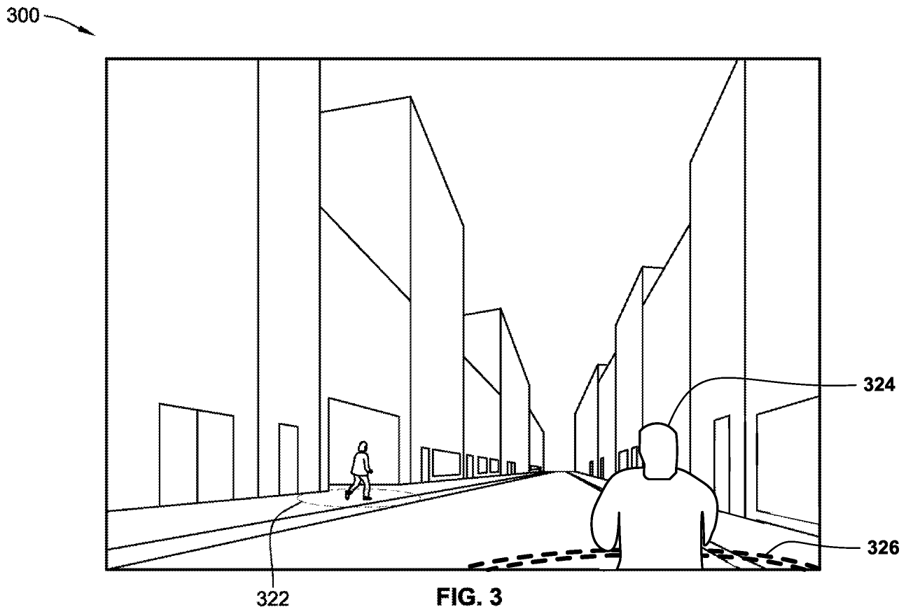

FIG. 3 is an illustration of a field of view 300 of a user including augmented reality overlays illustrating distance-based alerts, according to certain aspects of the present disclosure.

In the field of view 300, the user can see a first individual 320 and a second individual 324, both walking in an environment (e.g., down a street). In the field of view 300, the user can also see other objects, such as the street, sidewalks, buildings, windows, doors, and the sky.

Using acquired sensor data (e.g., camera data), the alert system can identify the first individual 320 as an entity in the environment and then identify the first individual 320 as a person. The alert system can then identify one or more high-risk activities associated with the first individual 320 (e.g., being within a certain distance of a person can be high-risk), such as based on the type of entity involved. The alert system can use the identified high-risk activity to provide an AR overlay, such as informational circle overlay 322. The information circle overlay 322 can be sized to indicate a threshold distance surrounding the first individual 320. For example, for a threshold distance of six feet, the information circle overlay 322 can be presented to cover a circular region around the first individual 320 having a radius of six feet.

In some cases, certain high-risk activities may be associated with different types of AR overlays. For example, while a high-risk activity involving proximity to an individual may make use of an overlay having the appearance of a distance circle surrounding the individual, a high-risk activity involving touching a potentially contaminated surface can make use of an overlay having the appearance of highlighting in the shape of the visible surface. Other types of AR overlays can be used with different types of high-risk activities and/or different types of entities.

The information circle overlay 322 can aid the user in easily and quickly identifying a safe distance to keep from the first individual 320. In some cases, the information circle overlay 322 is provided immediately upon the alert system identifying the first individual 320 as an entity and identifying the high-risk activity associated with the first individual 320. In some cases, however, the information circle overlay 322 can be presented only when the first individual 320 is within a threshold distance. This threshold distance for determining whether or not to display an informational overlay can be known as an attention threshold. The distance between the user and the first individual 320 can be determined using sensor data (e.g., data from a distance sensor).

Acquired sensor data can also allow the alert system to identify the second individual 324 as another entity in the environment and then identify the second individual 324 as a person. The alert system can then identify one or more high-risk activities associated with the second individual 324 (e.g., being within a certain distance of a person can be high-risk), such as based on the type of entity involved. The alert system can use the identified high-risk activity to provide an AR overlay, such as warning circle overlay 326. The warning circle overlay 326 can aid the user in easily and quickly identifying a safe distance to keep from the second individual 324. Additionally, because the second individual 324 is within a warning threshold distance, the warning circle overlay 326 can provide an indication that the second individual 324 is too close. For example, the warning circle overlay 326 can be similar to information circle overlay 322, but with different coloring, flashing, intensity, or other features designed to indicate urgency or importance. The distance between the user and the second individual 324 can be determined using sensor data (e.g., data from a distance sensor).

In some cases, an individual approaching the user can have no AR overlays (e.g., an information circle overlay 322 or warning circle overlay 326). For example, if an individual is identified (e.g., by the alert system) as belonging to a family, pod, or cohort associated with the user, the alert system may provide no AR overlays or may provide an AR overlay indicating that the individual is associated with a presumed safe group. In such cases, the alert system can use sensor data (e.g., camera data) to identify the individual and associated the individual with a list of safe individuals designated by the user.

In some cases, for an individual approaching the user, the alert system can present an information circle overlay 322 around the individual while the individual is at a distance from the user greater than the warning threshold distance (and optionally less than an informational threshold distance). When the distance between the user and the individual falls below the warning threshold distance, the alert system can present a warning circle overlay 326 around the individual.

In some cases, for an individual approaching the user, the alert system can dynamically adjust an AR overlay based on the distance between the user and the individual. In such cases, the dynamically adjusted AR overlay can be a continuously changing overlay, such as an overlay that continuously changes from blue at a safe distance (e.g., at a distance greater than the warning threshold) to red at an unsafe distance (e.g., at a distance closer than the warning threshold). In some cases, the dynamically adjusted AR overlay can be a stepwise (e.g., non-continuous) changing overlay, such as an AR overlay that changes when the distance between the user and the individual crosses multiple threshold distances. In such examples, the intensity of the AR overly can increase as the risk increases (e.g., as the individual comes closer to the user) so as to call more attention to the approaching individual.

In some cases, the alert system can predict a movement path of an individual (e.g., first individual 320 or second individual 324), such as based on recent historical positional data associated with the individual. For example, if the first individual 320 is seen walking away from the user in the field of view 300, the alert system can predict that the first individual 320 will continue to walk down the sidewalk in a direction away from the user. In some cases, additional AR overlays can be presented to indicate an individual's historical positional data (e.g., path of travel up to the current time) and/or an individual's predicted path (e.g., the path the individual is expected to take in the near future).

In some cases, the AR overlays presented to the user can be adjusted based on a predicted path of the individual. For example, if first individual 320 is predicted to walk away from the user, the alert system can provide an AR overlay with lower intensity or may provide no AR overlay, since the first individual 320 is likely not of high-risk to the user since the first individual 320 is walking away. However, if the first individual 320 turns around and starts walking towards the user, the alert system can provide an AR overlay with a higher intensity since there is a greater likelihood that the first individual 320 may come in close proximity to the user.

As used herein, intensity of an AR overlay or alert is meant to include a degree with which the AR overlay or alert is able to provoke a user's attention and/or indicate urgency. For example, a low intensity alert may be a subtle blue circle, whereas a high intensity alert may be a flashing red circle.

For illustrative purposes, FIG. 3 is described with reference to individuals in the user's field of view 300. However, aspects associated with individuals in FIG. 3 can also be applicable to other entities, such as other moving entities (e.g., moving cars, bikes, strollers, animals, and the like).

For illustrative purposes, FIG. 3 is described with reference to AR overlays presented in the field of view 500. These AR overlays can be alerts presented by the alert system. In some cases, other styles of AR overlays can be presented in addition to or instead of the AR overlays described with reference to FIG. 3. For example, instead of or in addition to the information circle overlay 322 and/or the warning circle overlay 326, the alert system can present an AR overlay with text or graphics indicating a distance between the user and the relevant individual. Additionally, in some cases, other alerts (e.g., audio alerts, haptic feedback, and the like) can be presented in addition to or instead of the AR overlays described with reference to FIG. 3. For example, when the second individual 324 approaches and/or first crosses the warning threshold distance, the alert system can provide an audio warning chime and/or a haptic feedback vibration, optionally from the direction of the second individual 324.

The alert system is able to present AR overlays within the field of view 300. In some cases, the alert system monitors only entities within the field of view 300. In some cases, however, the alert system monitors entities in a wider range, such as up to a full 360° circle around the user. In such cases, the alert system can provide an alert indicating a high-risk activity associated with an entity. An example of such an alert includes an AR overlay presented within the field of view 300 indicating the presence of a high-risk activity associated with an entity somewhere outside of the field of view 300 (e.g., an exclamation mark warning icon) and optionally indicating the direction of the entity (e.g., an arrow pointing to the right or left). Another example of such an alert includes an audio warning chime and/or haptic feedback vibration, optionally from the direction of the entity.

The various threshold distances used herein can be preset or dynamic. For example, a preset threshold distance can be set at 6′ to promote maintaining a six-foot distance between the user and another individual. As another example, a dynamic threshold distance can be adjusted based on environmental factors (e.g., increase or decrease the threshold distance based on humidity, temperature, airflow, or identified environment), posted rules or recommendations (e.g., a sign recommending 10 foot distance as detected by a sensor of the alert system), a condition of the user (e.g., a user with a high respiration rate and/or heart rate may be at higher risk when in close proximity to another, so the threshold distances may be increased), or other factors. In some cases, threshold distances can be based on user preference, laws and/or rules, environmental factors, and/or received preferences from a remote device (e.g., received preferences from a remote alert system worn by first individual 320).

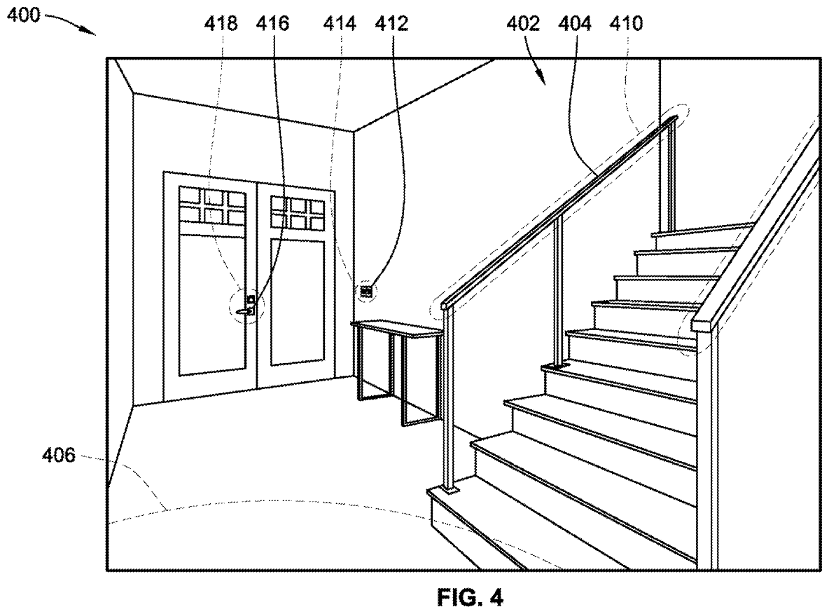

FIG. 4 is an illustration of a field of view 400 of a user including augmented reality overlays illustrating high-touch alerts, according to certain aspects of the present disclosure. Field of view 400 can be the portion of the environment surrounding the user that is visible to the user. When the user is making use of an alert system as disclosed herein, such as alert system 100 of FIG. 1, the user can be presented with AR overlays. Any suitable AR overlays can be provided, such as those disclosed herein, as well as other alerts as disclosed herein. As depicted in FIG. 4, the AR overlays are generally related to high-touch. While other AR overlays might be presented in the field of view 400 depicted in FIG. 4, for illustrative purposes, only select AR overlays are shown in FIG. 4.

The user is in an environment, such as a foyer of a home. In the field of view 400, the user can see a portion of the environment, including a stairway 402 including steps and a handrail 404, a table, a light switch 412, a doorway, doors, a door handle 416, walls, a ceiling, and a floor.

In some cases, the alert system can present a user circle overlay 406, such as on the ground around the user. The user circle overlay 406 can be presented to indicate a region surrounding the that is designated by a particular threshold distance. For example, for a threshold distance of six feet, the user circle overlay 406 can be presented to cover a circular region around the user having a radius of six feet. The user circle overlay 406 can be used to easily and quickly identify a safe region around the user and can help the user maintain a safe distance from other entities (e.g., individuals, animals, fomites, and the like) in the environment.

The alert system can identify different entities within the environment, such as entities within the field of view 400. For example, the alert system can identify the stairway 402, including steps and the handrail 404. When identifying the handrail 404 as an entity, the alert system can identify that particular entity as a high-touch surface (e.g., a surface likely to have been contacted by others or by pathogen-carrying objects). Identifying the entity as a high-touch surface can include identifying the entity as a surface, determining a type entity (e.g., a hand support and/or a handrail), determining a height of the surface (e.g., at a height near the hands of an individual passing the surface or at a height within reach of the hands of an individual passing the surface), or other such information. When the alert system identifies the handrail 404 as a high-touch surface, the alert system can present an AR overlay calling attention to the handrail 404, such as by presenting a surface highlighting graphic 410. The surface highlighting graphic 410 can take shape of the high-touch surface (e.g., be shaped to exactly cover the handrail 440 in the field of view 400), can be slightly larger than the high-touch surface (e.g., be shaped to cover the handrail 404 and a region around the handrail 404 in the field of view 400; or be shaped to surround the handrail 404, such as by a rounded rectangle or other shape), or can be otherwise shaped to call attention to the handrail 404. In some cases, the alert system only presents the surface highlighting graphic 410 once the user is within a threshold distance of the handrail 404. This threshold distance can be similar to the attention threshold described with reference to FIG. 3. Because the steps of the stairway 402 are not associated with a high-risk activity (e.g., walking on steps does not present a high risk, whereas touching a handrail 404 with one's hands can present a high risk), the alert system may provide no AR overlay associated with the steps, even if the alert system identifies the steps as an entity within the environment.

The light switch 412 and door handle 416 can also be identified by the alert system. The alert system can determine that both the light switch 412 and door handle 416 are high-touch surfaces. Thus, the alert system can provide respective surface highlighting graphic 414 and surface highlighting graphic 418 at or around the light switch 412 and door handle 416, respectively. Thus, when the user is looking at field of view 400 as depicted in FIG. 4, the high-touch surfaces (handrail 404, light switch 412, and door handle 416) are highlighted by respective AR overlays (surface highlighting graphics 410, 414, 418).

In some cases, one or more sensors of the alert system can detect movement of the user's extremities, such as movement of the user's hand. In such cases, one or more AR overlays or other alerts can be presented by the alert system when the user's hand approaches the high-touch surface. For example, the surface highlighting graphic 410 may only appear or may change in appearance (e.g., to indicate greater urgency), or another alert can be given, when the alert system detects that the user's hand is moving to make contact with the handrail 404.

As depicted in FIG. 4, the AR overlays for calling attention to high-touch surfaces are illustrated as shapes surrounding the high-touch surface. In some cases, the AR overlay would provide a translucent or opaque region of color, which can call the user's attention to the high-touch surface. In some cases, the AR overlay would provide a circle around the high-touch surface, without blocking the high-touch surface itself. In some cases, however, other AR overlays can be used. For example, high-touch surfaces can be indicated with icons (e.g., a circle with a slash icon indicating do not touch or an exclamation mark indicating warning), text (e.g., a message saying “avoid touching with hands”), or the like. Further, in some cases, other alerts (e.g., audio alerts and/or haptic feedback alerts) can be provided instead of or in addition to AR overlays.

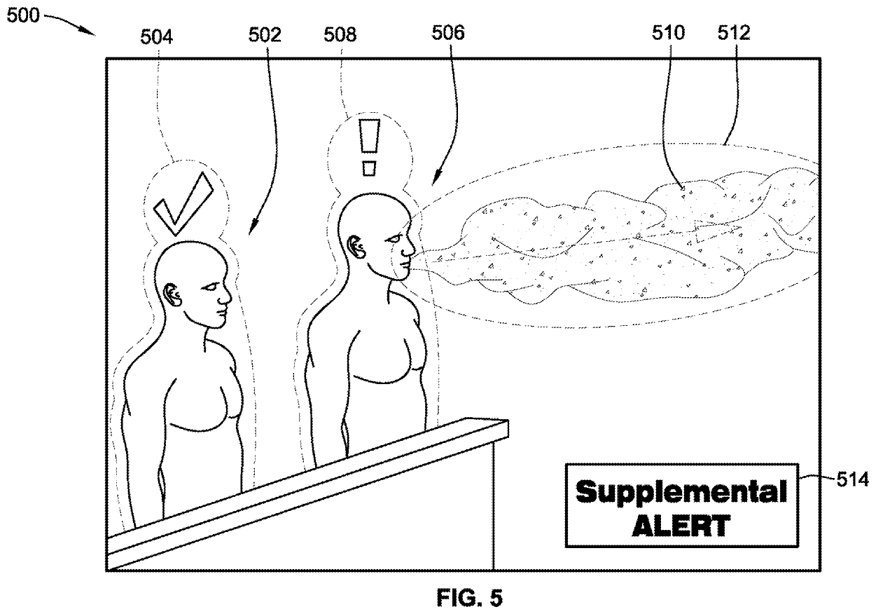

FIG. 5 is an illustration of a field of view 500 of a user including augmented reality overlays illustrating high-risk alerts, according to certain aspects of the present disclosure. Field of view 500 can be the portion of the environment surrounding the user that is visible to the user. When the user is making use of an alert system as disclosed herein, such as alert system 100 of FIG. 1, the user can be presented with AR overlays. Any suitable AR overlays can be provided, such as those disclosed herein, as well as other alerts as disclosed herein. As depicted in FIG. 5, the AR overlays are generally related to high-risk alerts. While other AR overlays might be presented in the field of view 500 depicted in FIG. 5, for illustrative purposes, only select AR overlays are shown in FIG. 5.

The user may be in an environment, such as an outdoor space (e.g., uncovered field, sidewalk of a street with buildings, a covered gazebo, and others) or an indoor space (e.g., a room, an entryway, a conference room, an airport, a vehicle, and others). The user can see a first individual 502 and a second individual 506 in the field of view 500.

In some cases, one or more sensors in the alert system can be used to identify a type of environment. For example, the one or more sensors can be used to identify that the user is in an outdoor space, an indoor space, a car, a bus, a large room, a small room, or types of environments. In some cases, the one or more sensors can be used to determine information about the environment, such as an approximate size of the room (e.g., square footage and/or volume), an approximate number of people in the room, ambient temperature, humidity, and the like. In some cases, the one or more sensors can include determining a geolocation of the user to help identify the environment or determine more information about the environment.

In some cases, the alert system can access one or more databases of rules that are based on a particular environment (e.g., a particular store at a particular street address), a particular type of environment (e.g., any restaurant), or information about the environment (e.g., approximate square footage or volume). Rules can be any suitable rules, such as user-defined rules, cohort-defined rules (e.g., rules for members of the cohort, such as family-specific rules for all family members or company-specific rules for all employees of the company), business-defined rules (e.g., a place of business's own rules, such as a restaurant that wants to keep space between occupants of at least eight feet), laws (e.g., state or federal laws), and/or regulations (e.g., local, state, or federal regulations or other regulations). Rules can establish certain parameters for different environments, which can be leveraged by the alert system to provide alerts and/or update parameters of the alert system (e.g., threshold distances). In some cases, a commercial establishment can have an occupancy rule that defines a maximum allowed occupancy of the establishment. The occupancy rule can be used to trigger alerts and/or adjust parameters of the alert system.

In some cases, information about the environment can include one or more rules or recommendations posted at the environment, which can be detected by the one or more sensors. For example, a restaurant can post a placard indicating that patrons should maintain six feet of distance, in which case the alert system can identify and interpret the placard to set its warning threshold distance at six feet. Such information can be posted at an environment using visible techniques (e.g., signage detectable by a camera) or non-visible techniques (e.g., a radio frequency signal detectable by a radio frequency sensor or interface of the alert system).

Based on the identified type of environment or other information about the environment, the alert system can present alerts (e.g., audio alerts, haptic feedback, or AR overlays) to the user. For example, when entering a small store from a street, the alert system can alert the user (e.g., present an alert in the form of an AR overlay of text saying “Caution: Confined Space. Do Not Linger” and/or an AR overlay of a countdown timer based on a desired time not to exceed lingering within the confined space). These types of alerts can be presented as an AR overlay, such as supplemental alert 514.

In some cases, based on the identified type of environment or other information about the environment, the alert system can modify parameters of its operation. For example, based on the type of environment or other information about the environment, the alert system can change the threshold distance for providing a warning about the proximity of another individual. In an example, the alert system may have a warning threshold distance set at six feet by default, but when entering a restaurant that has established its own minimum distance of eight feet, the alert system can automatically adjust its warning threshold distance to eight feet while the user is in that environment.

In some cases, the alert system can be used to detect symptom-related actions. Symptom-related actions can be any actions performed by an individual that may be indicative that the individual may have transmittable pathogens. For example, an individual coughing or sneezing can be identified from the sensor data of the one or more sensors of the alert system. Based on the frequency, duration, presence, and/or intensity of the symptom-related action, the alert system can determine whether or not the individual presents a risk (e.g., whether or not coming into close proximity of the individual is a high-risk activity). In some cases, the alert system can calculate an infection risk score based on the detection of one or more symptom-related actions. The alert system can provide alerts and/or modify parameters of its operation based on the infection risk score and/or the determination about whether or not the individual presents a risk. In some cases, symptom-related actions can include an elevated temperature, which can be detected from one or more sensors of the alert system, such as a pyrometer. Other examples of symptom-related actions can include gait, sweating, facial expression, and others.

In an example first individual 502 may exhibit no or few symptom-related actions, in which case the alert system may provide no alerts or informational alerts (e.g., informational circle overlay 322 of FIG. 3). For example, the alert system can present a safe overlay graphic 504 adjacent the first individual 502. The safe overlay graphic 504 can be an AR overlay that is indicative that the level of risk associated with interacting with the first individual 502 is below a threshold (e.g., no detectable symptoms are visible).

However, second individual 506 is exhibiting symptom-related actions, in this case sneezing. The one or more sensors of the alert system can detect the symptom-related action (e.g., via camera data and/or audio data) and determine that the individual is associated with a high risk. For example, detection of repeated symptom-related actions within a period of time can be used to calculated an infection risk score. Based on determining that the individual is associated with a high risk and/or has an infection risk score above a threshold, the alert system can present an alert and/or modify alerts related to the second individual 506. In an example, the alert system can present an unsafe overlay graphic 508 adjacent the second individual 506. The unsafe overlay graphic 508 can be an AR overlay that is indicative that the level of risk associated with interacting with the second individual 506 is above a threshold (e.g., sufficient detectable symptoms are visible). In another example, the alert system can present an informational circle overlay around the second individual 506 with a greater radius than an informational circle overlay over the first individual 502.

In some cases, detection of symptom-related actions of an individual within a certain distance from the user can be used to modify parameters of the alert system. For example, if the detection of symptom-related actions can cause the alert system to increase its warning threshold distance for a period of time or until the individual associated with the symptom-related actions is sufficiently far away from the user.

In some cases, certain symptom-related actions can be highlighted or otherwise called to attention by an alert, such as an AR overlay. In an example, second individual 506 is seen sneezing. During the sneeze, particles 510 (e.g., droplets and/or aerosolized particles) can be expelled from the mouth and/or nose of the second individual 506 and by carried over a distance (e.g., approximately 6 feet in many cases). Particles 510 may be invisible to the naked eye. Upon detecting a sneeze, the alert system can present an AR overlay, such as a plume graphic 512, that can indicate the path and/or direction of the particles 510. Similar AR overlays can be associated with and triggered by other symptom-related actions.

Depicted in the field of view 500 is a supplemental alert 514. The supplemental alert 514 is an AR overlay that can be associated with an entity in the environment and/or a high-risk action. In some cases, the supplemental alert 514 can provide information about the environment, such as a text overlay providing the approximate size of the room in which the user is located. In some cases, the supplemental alert 514 can provide information about a detected symptom-related action, such as a text overlay indicating that an individual nearby (e.g., second individual 506) has sneezed. In some cases, the supplemental alert 514 can provide information about a high-risk action, such as a warning when the user's hand approaches a high-touch surface or when the user's hand approaches the user's face. In some cases, the supplemental alert 514 can provide instructions and/or advice to minimize risk of infection, such as instructions to not linger in an enclosed space or instructions to wear a face covering. The supplemental alert 514 can take other forms, as well.

In some cases, the alert system can be used to trigger an alert associated with a face covering (e.g., a facemask or face shield), which can be presented as a supplemental alert 514. Examples of such alerts include an alert to wear a face covering (e.g., wear a face covering when an individual is within a threshold distance of the user), an alert to clean or replace a face covering (e.g., if the face covering has been worn for an extended period of time), an alert to avoid touching a face covering (e.g., if the user's hand is identified as approaching the face covering), and an alert to remove a face covering (e.g., if the user leaves a public environment and goes to a private environment). Other types of alerts can be provided.

In some cases, in addition to or instead of providing an alert associated with a face covering as a supplemental alert 514, the alert system can initiate automatic deployment of a face covering. In an example, when the user moves from an environment where no face covering is needed to an environment where a face covering is needed or desired, the alert system can automatically deploy a face covering. Deploying a face covering can include lowering or raising a face shield, lowering or raising a facemask, deploying an air curtain (e.g., a stream of air to urge particles away from the user's face), or otherwise protecting the user's face. In some cases, a face covering can be deployed in response to a nearby action, such as an individual approaching the user and coming within a threshold distance or an individual sneezing in the direction of the user.

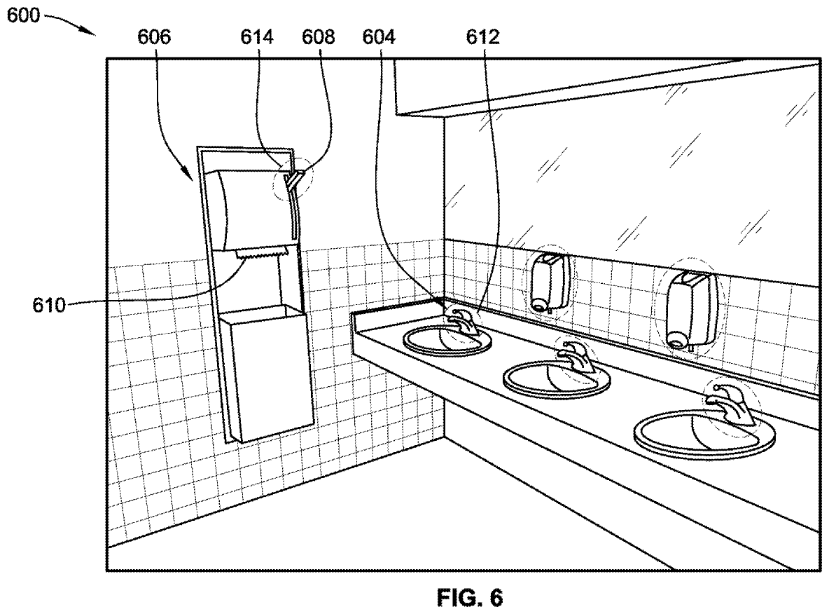

FIG. 6 is an illustration of a field of view 600 of a user including augmented reality overlays illustrating high-risk alerts in a restroom, according to certain aspects of the present disclosure. Field of view 600 can be the portion of the environment surrounding the user that is visible to the user. When the user is making use of an alert system as disclosed herein, such as alert system 100 of FIG. 1, the user can be presented with AR overlays. Any suitable AR overlays can be provided, such as those disclosed herein, as well as other alerts as disclosed herein. As depicted in FIG. 6, the AR overlays are generally related to high-risk alerts. While other AR overlays might be presented in the field of view 600 depicted in FIG. 6, for illustrative purposes, only select AR overlays are shown in FIG. 6.

The user can be in an environment that is a bathroom, such as a public bathroom. The user may see, in the field of view 600, sinks, faucets with faucet knobs 604, and a towel dispenser 606.

The alert system can identify the faucet knobs 604 and touch bar 608 of the towel dispenser 606 as high-touch surfaces. The alert system can provide AR overlays on the high-touch surfaces, such as a surface highlighting graphic 612 on the faucet knobs 604 and a surface highlighting graphic 614 on the towel bar 608 of the towel dispenser 606. The remainder of the towel dispenser 606 and the fresh towel 610 that is being dispensed are not highlighted, since they would not be identified as high-touch surfaces. However, in some cases, the entire towel dispenser 606 can be highlighted. The alerts used for the high-touch surfaces in FIG. 6 can be similar to the alerts used for high-touch surfaces in FIG. 4.

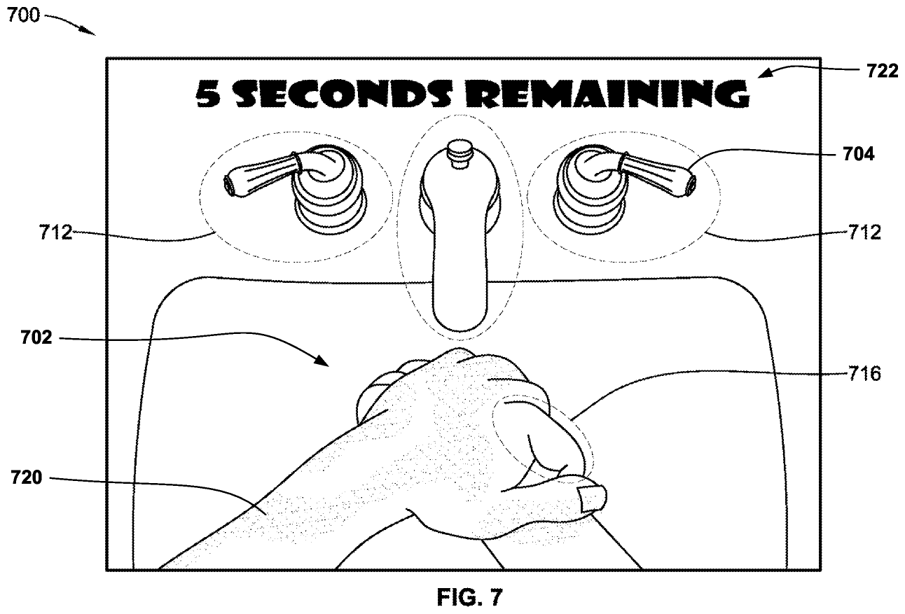

FIG. 7 is an illustration of a field of view 700 of a user including augmented reality overlays illustrating alerts associated with cleaning actions, according to certain aspects of the present disclosure. Field of view 700 can be the portion of the environment surrounding the user that is visible to the user. When the user is making use of an alert system as disclosed herein, such as alert system 100 of FIG. 1, the user can be presented with AR overlays. Any suitable AR overlays can be provided, such as those disclosed herein, as well as other alerts as disclosed herein. As depicted in FIG. 7, the AR overlays are generally related to alerts associated with cleaning actions. While other AR overlays might be presented in the field of view 700 depicted in FIG. 7, for illustrative purposes, only select AR overlays are shown in FIG. 7.

The user can be in an environment that is a bathroom, such as a public bathroom. The user may see, in the field of view 700, a sink, a faucet with faucet knobs 704, and the user's hands 702.

In some cases, the alert system can provide interactive feedback associated with activities that lower risk of infection, such as cleaning. For example, the alert system can provide AR overlays to provide instructions, timers, and/or other information associated with cleaning an object, such as cleaning a table or cleaning the user's hands. In some cases, the AR overlay can highlight regions of the object that have or have not yet been sufficiently cleaned. The AR overlay can thus be used to differentiate cleaned (e.g., washed) and yet-to-be-cleaned (e.g., unwashed) portions of an object (e.g., hands).

In the depicted example, when the user's hands 702 are in the user's field of view 700, the alert system can present a washing completion graphic 716 over the user's hands 702. The user 702 can be in the process of washing their hands 702 with soap 720, but has not fully cleaned all regions of the user's hands 702 with the soap 720. The washing completion graphic 716 can be a translucent or opaque graphic that indicates portions of the user's hands 702 that have been not yet been washed. The washing completion graphic 716 highlights regions of the user's hands 702 that have not yet been washed (e.g., are devoid of soap 720), in which case the user is able to know their hands 702 have not yet been sufficiently washed and can quickly and easily identify which regions need attention. In other examples, a washing completion graphic can instead depict portions of the user's hands that have already been washed, in which case the user can continue washing until no more washing completion graphic covers any portion of the user's hand.

The alert system can determine regions of the object that have been washed based on sensor data (e.g., camera data). The alert system can track movement and position of the user's hands 702 to identify regions of the surface of the user's hands 702 that have and have not been washed. While described with reference to soap 720, similar washing completion graphics can be used when applying other cleaning agents, such as hand sanitizer.

In some cases, especially where sufficiency of cleaning is related to time (e.g., a suggested time for exposing a surface to a cleaning agent to properly clean the surface or a suggested time for how long to wash one's hands), a timer overlay (e.g., timer graphic 722) can be presented. For example, when the user begins washing their hands 702, the alert system can determine that the user is engaging in handwashing based on sensor data (e.g., camera data and/or audio data) and start a timer (e.g., a countdown timer or a stopwatch counting up). The alert system can present the timer as a timer graphic 722 adjacent the user's hands 702. Thus, the timer graphic 722 can facilitate the user in engaging in the cleaning activity for a sufficient amount of time.

FIG. 8 is a flowchart depicting a process 800 for protecting against infectious disease, according to certain aspects of the present disclosure. Process 800 can be performed by any suitable alert system, such as alert system 100 of FIG. 1.

At block 802, sensor data is collected from one or more sensors on a mobile platform. The mobile platform can be worn by a user. The user can be located in an environment. The sensor data can be associated with the environment in which the user is located. Collecting sensor data can include collecting camera data (e.g., still or video images, including live still or video images), collecting audio data, collecting distance information (e.g., data from a LiDAR system), or collecting any other suitable data.

At block 804, the sensor data can be analyzed to identify a set of entities in the environment. The set of entities can include any number of entities detectable to the alert system. In some cases, the set of entities includes all entities in the environment. In other cases, the set of entities includes entities in the environment that fall within and/or near the user's field of view. The types of objects in an environment that the alert system identifies as being an entity can be predetermined, such as by being trained into a model (e.g., a neural network model, such as a deep learning model). In an example, entities can be individuals and potential fomites (e.g., high-touch surfaces) in an environment. The alert system can identify some or all of the entities in an environment, even including entities that may not pose any risk or may only pose a low risk. In some cases, the alert system can be trained such that it only identifies entities that may pose a risk or may pose a sufficient risk. In some cases, a user can select from differently trained models, which can have the alert system focus on and/or ignore different types of entities (e.g., some models may identify only individuals that show signs of symptoms of a disease, whereas other models may identify all individuals). In some cases, only moving entities (e.g., entities moving with respect to the user's frame of reference) are identified.

At block 806, the alert system can determine high-risk action(s) associated with the set of entities. Some (e.g., only entities in and/or near the user's field of view) or all of the entities in the environment can be analyzed to determine the high-risk action(s). Determining a high-risk action for an entity can include identifying an entity type (e.g., individual, surface, or others) for the entity and selecting from one or more activities associated with the entity. For example, if the entity is determined to be an individual, the high-risk action can include being in close proximity to the individual. Thus, the alert system can identify someone as an entity and then determine that a high-risk action exists because that entity is an individual.

In some cases, determining a high-risk action exists can include determining (e.g., calculating) an infection risk score associated with the entity. If the infection risk score is above a threshold, the high-risk action can exist. Determining an infection risk score can include analyzing the sensor data collected at block 802 to identify information about the entity. For example, an infection risk score can be based, at least in part, on a measured temperature of an individual. In another example, an infection risk score can be based, at least in part, on identifying a type of material a surface is made from (e.g., some surface materials may be more prone to collecting and/or releasing pathogens than others). One or more models can be trained to identify a type of material based on the available sensor data (e.g., camera data, pyrometer data, acoustic data).

In some cases, determining a high-risk action exists can include identifying a symptom-related action associated with an entity. For example, if an individual sneezes, the detected sneeze can be registered as a symptom-related action, which can cause the entity to be identified as high-risk and/or can increase an infection risk score associated with the entity.

Determining a high-risk action can otherwise or additionally occur as disclosed elsewhere, herein.

At block 808, a distance between a reference location and at least one entity of the set of entities can be measured. The reference location can be the location of the user, the location of the alert device, or a location of the one or more sensors used to collect the sensor data at block 802. Measuring the distance at block 808 can involve using the sensor data, such as using depth measurements. Depth measurements can be used to determine a distance between the user and a surface of the at least one entity. For example, a LiDAR system can establish a distance between the user and an individual. In some cases, measuring distance at block 808 can include measuring distance to all entities of the set of entities. In some cases, however, distance measured at block 808 is specifically for an entity identified as being associated with the high-risk action from block 806.

At block 810, the alert system can generate an alert when the measured distance from block 808 drops below a threshold distance. In response to generating the alert at block 810, the alert can be presented on the mobile platform at block 812. In some cases, the alert generated at block 810 can be based on the type of high-risk action, the type of entity, and/or the type of threshold distance that the measured distance drops below. Presenting the alert at block 810 can include presenting the alert as an AR overlay. In some cases, presenting the alert at block 810 can include presenting an audio alert and/or a haptic alert.

In an example, the alert system can identify that the entity is an individual and the high-risk action is proximity to the individual. In such a case, if the individual is determined to be at a distance within an informational threshold distance, the alert generated and presented can be a AR overlay of a circle around the individual indicating a safe zone around the individual. If that same individual is determined to be at a distance within a warning threshold distance, the alert generated and presented can be an AR overlay of an urgent circle (e.g., flashing, filled-in with red, or otherwise attention-seeking) indicating the individual has breached (or is about to breach) a save zone around the user.

In another example, if the high-risk action is identified to be contacting a high-touch surface, the alert generated and presented can be an AR overlay highlighting the high-touch surface, calling attention to the surface and indicating that it is a high-touch surface.

In some cases, the process 800 can optionally include storing information associated with the high-risk action at block 814. This information can be stored when the measured distance from block 808 falls below a threshold distance (e.g., an event), which can be the same threshold distance from block 810 or a different threshold distance. This information can include the type of high-risk action, date information, time information, location information, environmental information, information about the user (e.g., whether the user was wearing gloves), and sensor data (e.g., a video clip of the event). In some cases, the information stored at block 814 is only for events where the high-risk action occurred (e.g., where an individual crossed into the user's safe zone). In other cases, the information stored at block 814 can additionally include events where the high-risk action almost occurred (e.g., where an individual was close to crossing into the user's safe zone).

In some cases, the process 800 can optionally include generating, at block 816, a summary of the detected high-risk actions based on the information stored at block 814. Examples of summaries include a list of the high-risk actions, a list of entities associated with the high-risk action, a time-lapse of all events during a time period, a score (e.g., a cleanliness score or compliance score), or any combination thereof.

The blocks of process 800 can be performed in any suitable order, as appropriate. For example, information stored at block 814 can occur before or after an alert is generated at block 810. In some cases, blocks of process 800 can be repeated and/or be performed continuously. For example, collecting sensor data at block 802 can occur continuously while the alert system is in user. In some cases, additional or fewer blocks may be used with process 800.

In some cases, process 800 can include generation of additional alerts, including any combination of the alerts described herein.

FIG. 9 is a flowchart depicting a process 900 for determining a high-risk action associated with a person, according to certain aspects of the present disclosure. Process 900 can be performed by any suitable alert system, such as alert system 100 of FIG. 1. In some cases, process 900 can be performed as part of block 806 of process 800 of FIG. 8.

At block 902, an infection risk score associated with the individual is determined. Determining an infection risk score can include obtaining sensor information associated with the individual and calculating an infection risk score using the sensor information. In some cases, determining an infection risk score includes measuring a temperature of the individual at block 904. The measured temperature can be compared to a threshold value and/or otherwise used to calculate or adjust the infection risk score. For example, an individual having a body temperature above 39° C. (102.2° F.) may be presumed to be ill, which can raise the infection risk score associated with the individual. In some cases, an infection risk score can be calculated by applying the measured temperature to a formula.

In some cases, determining an infection risk score can include detecting a symptom-related action at block 906. Detecting a symptom-related action can include analyzing sensor data (e.g., camera data, audio data, and the like) to detect when the individual exhibits a symptom-related action. For example, when an individual sneezes, process 900 may analyze sensor data, including a video feed from a camera and/or an audio feed from a microphone, to detect the sneeze. In some cases, the sensor data can be used to predict a future occurrence of a sneeze, such as if the individual makes movements, facial expressions, and/or sounds that may be indicative of an oncoming sneeze. In some cases, the sensor data can be used to identify occurrence of a sneeze, such as based on head movements of the individual and accompanying sneezing sounds. Detecting a symptom-related action can include applying the sensor data to a model, such as a deep neural network, trained to identify symptom-related actions generally and/or specific symptom-related action. In some cases, the infection risk score can be calculated and/or adjusted based on the number of symptom-related actions over time, the intensity of one or more symptom-related actions, the frequency of symptom-related actions (e.g., the time between the current symptom- related action and the previous symptom-related action), the type of symptom-related action, or any combination thereof. Other information associated with the symptom-related action can be used to calculate and/or adjust the infection risk score.

At block 908, the process 900 can include identifying that interaction with the individual is a high-risk action when the infection risk score is above an infection risk threshold. If the infection risk for a particular individual does not meet or exceed the threshold, the process 900 may decide that interaction with that individual does not amount to a high-risk action. However, if the infection risk is sufficiently high, it may be determined that interaction with that individual is a high-risk action. In some cases, the infection risk threshold can be preset, such as factory-set (e.g., set during manufacturing), business-set (e.g., set by an employer), group-set (e.g., set by a group, such as a school), or user-selectable. In some cases, the infection risk threshold can be dynamically adjusted based on sensor data, such as dynamically adjusted based on the user's location, the type of environment in which the user is located, the number of individuals in the environment, or other such factors.

At optional block 910, the infection risk score can be used to update a threshold distance, such as the threshold distance used to determine when to generate an alert or a threshold distance used to otherwise generate the alert (e.g., a threshold distance used to determine a size of circle to present around an individual). Thus, the infection risk score associated with a user can be used to dynamically adjust parameters of the alert system. For example, if an individual's infection risk score is low, the threshold distance for that individual may be lowered and thus the AR overlay graphic of a circle around that individual may be smaller. However, if an individual's infection risk score is high, the threshold distance for that individual may be increased and thus the AR overlay graphic of a circle around that individual may be larger.

In some cases, other actions can be taken and/or other parameters adjusted based on the infection risk score of an individual. For example, when it is determined that the infection risk score for an individual is above an infection risk threshold, the alert system can present an AR overlay in the form of a graphic adjacent the individual in the user's field of view that indicates the user is or may be ill.

The blocks of process 900 can be performed in any suitable order, as appropriate. For example, updating the threshold distance at block 910 can occur before or after identifying the interaction with the person is the high-risk action at block 908. In some cases, blocks of process 900 can be repeated and/or be performed continuously. For example, measuring a temperature of the individual at block 904 can occur repeatedly to dynamically update the infection risk score associated with the individual. In some cases, additional or fewer blocks may be used with process 900.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the invention. Thus, the breadth and scope of the present invention should not be limited by any of the above described embodiments.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

1. A method for protecting against transmission of infectious diseases comprising:
collecting sensor data associated with an environment using one or more sensors on a mobile platform associated with a user located within the environment, wherein a location of the user within the environment defines a reference location;
identifying a set of entities in the environment using the sensor data, the set of entities including one or more entities;
determining a high-risk action associated with the set of entities using the sensor data, wherein the high-risk action is associated with an interaction between the user and at least one entity of the set of entities;
measuring a distance, using the sensor data, between the at least one entity of the set of entities and the reference location;
generating an alert when the measured distance drops below a threshold distance, wherein the alert is indicative of the high-risk action; and
presenting the alert on an interface associated with the mobile platform.

2-3. (cancelled)

4. The method of claim 1, wherein the at least one entity is a person in the environment.

5. The method of claim 4, wherein determining the high-risk action includes:
determining an infection risk score associated with the person using the sensor data; and
identifying that interaction with the person is the high-risk action when the infection risk score is above an infection risk threshold.

6. (canceled)

7. The method of claim 5, wherein determining the infection risk score includes:
detecting one or more symptom-related actions based on the sensor data; and
calculating the infection risk score based on the detected one or more symptom-related actions.

8. The method of claim 5, further comprising updating the threshold distance based on the infection risk score.

9-10. (canceled)

11. The method of claim 1, wherein the at least one entity is a surface, and wherein determining the high-risk action includes determining that the surface is a high-touch surface.

12. The method of claim 1, further comprising determining an environmental condition using the sensor data, wherein determining the high-risk action is further based on the environmental condition.

13. The method of claim 1, further comprising:
determining an environment type of the environment using the sensor data; and
setting the threshold distance based on the environment type.

14. The method of claim 1, further comprising:
determining a geolocation associated with the environment;
accessing a rule based on the geolocation; and
setting the threshold distance based on the rule.

15. The method of claim 1, further comprising:
identifying rule signage using the sensor data, wherein the rule signage is a sign present in the environment indicative of a desired amount of distancing between individuals;
determining a rule based on the rule signage; and
setting the threshold distance based on the rule.

16. (canceled)

17. The method of claim 1, wherein presenting the alert includes presenting information about reducing a risk of infection after engaging in the high-risk action, wherein the information about reducing the risk of infection includes an instruction to deploy a facial covering, and wherein the method further comprises:
detecting deployment of the facial covering using the sensor data; and
ceasing to present the instruction to deploy the facial covering in response to detecting deployment of the facial covering.

18-20. (canceled)

21. The method of claim 1, further comprising automatically deploying a facial covering when the determined distance between the at least one entity and the reference location drops below the threshold distance.

22. (canceled)

23. The method of claim 21, wherein deploying the facial covering includes i) moving a face shield from a stowed position to a deployed position, wherein the face shield covers at least a portion of a face of the user when in the deployed position; ii) moving a facemask from a stowed position to a deployed position, wherein the facemask covers a mouth and nose of the user when in the deployed position; iii) generating an air curtain around a portion of a face of the user; or iv) any combination of i-iii.

24-25. (canceled)

26. The method of claim 1, further comprising:
storing information associated with the high-risk action in response to the measured distance dropping below the threshold distance; and
generating a summary of detected high-risk actions that occurred within a period of time, wherein generating the summary includes accessing the stored information associated with the high-risk action, wherein the summary of detected high-risk actions includes i) a score based on a count of the detected high-risk actions; ii) a listing of types of high-risk actions associated with each of the detected high-risk actions; iii) a listing of types of entities associated with each of the detected high-risk actions; or iv) any combination of i-iii.

27-32. (canceled)

33. The method of claim 26, wherein the detected high-risk actions includes high-risk actions associated with entities not within a predefined cohort of entities.

34-52. (canceled)

53. The method of claim 1, wherein presenting the alert includes presenting an augmented reality alert on a display device of the mobile platform, wherein presenting the augmented reality alert includes presenting at least a portion of an entity ring centered at the at least one entity, and wherein a radius of the entity ring is indicative of the threshold distance or a supplemental threshold distance from a center of the at least one entity.

54. The method of claim 1, wherein presenting the alert includes presenting an augmented reality alert on a display device of the mobile platform, wherein presenting the augmented reality alert includes presenting at least a portion of a reference ring centered at the reference location, and wherein a radius of the reference ring is indicative of the threshold distance or a supplemental threshold distance from a center of the at least one entity.

55. (canceled)

56. The method of claim 55, wherein presenting the alert includes presenting an augmented reality alert on a display device of the mobile platform, wherein the high-risk activity is associated with particulates projected from the at least one entity, wherein the method further comprises determining an expected path of travel associated with the particulate projection, and wherein presenting the augmented reality alert includes indicating the path of travel associated with the projected particulates.

57-58. (canceled)

59. The method of claim 57, wherein presenting the alert includes presenting an augmented reality alert on a display device of the mobile platform, the method further comprising presenting an additional augmented reality alert associated with the at least one entity before the determined distance drops below the threshold distance, wherein the at least one entity includes a moving entity, the method further comprising calculating a probable path of the at least one entity, wherein the additional augmented reality alert is indicative of the probable path of the at least one entity.

60. (canceled)

61. The method of claim 1, wherein presenting the alert includes presenting an augmented reality alert on a display device of the mobile platform, the method further comprising presenting an additional augmented reality alert associated with the at least one entity before the determined distance drops below the threshold distance, wherein the additional augmented reality alert:
i) is indicative that the at least one entity includes a high-touch surface;
ii) is indicative of a need to perform handwashing;
iii) is indicative of a percentage of completion of handwashing;
iv) is based on an accessed health rating or cleanliness rating of a commercial establishment associated with the environment;
v) is based on a number of individuals in the environment determined using the sensor data;
vi) is indicative that a threshold number of individuals for the environment has been exceeded;
vii) is indicative of a path determined, using the sensor data, between the entities of the set of entities, wherein the path is calculated to maintain at least the threshold distance between the path and each entity of the set of entities;
viii) includes an overlay indicating a point on a floor of the environment that is within a queue or at an end of the queue, the queue comprising a subset of the set of entities, wherein the point on the floor is at least the threshold distance spaced apart from a nearest entity within the subset of entities comprising the queue; or
ix) any combination of i-viii.

62-80. (canceled)
